(19) United States
(12) Patent Application Publication
(10) Pub. No.: US 2025/0249221 A1
(43) Pub. Date: Aug. 7, 2025

(54) DRUG ELUTING APPARATUS AND METHOD OF DELIVERING DRUG

(71) Applicant: TERUMO MEDICAL CORPORATION, Somerset, NJ (US)

(72) Inventors: Sarah Gettinger, West Chester, PA (US); Seiichi Otsubo, Newark, NJ (US)

(73) Assignee: TERUMO MEDICAL CORPORATION, Somerset, NJ (US)

(21) Appl. No.: 19/120,170

(22) PCT Filed: Mar. 20, 2023
(86) PCT No.: PCT/US2023/015679

§ 371 Date: Apr. 10, 2025

Related U.S. Application Data
(60) Provisional application No. 63/326,006, filed on Mar. 31, 2022.

(51) Int. Cl. A61M 25/10

(52) U.S. Cl. CPC A61M 25/10; A61M 2025/105; A61M 2025/1056

(57) ABSTRACT

A drug eluting apparatus includes: a handle including: a balloon inflation port configured to receive an inflation fluid, and a drug port configured to receive a drug; a shaft connected to the handle, the shaft including: a balloon inflation lumen configured to receive the inflation fluid from the balloon inflation port, a drug lumen configured to receive the drug from the drug port, and a guidewire lumen configured to receive a guidewire and allow the guidewire to extend out of a distal portion of the shaft; a balloon attached to the distal portion of the shaft and configured to receive the inflation fluid from the balloon inflation lumen; and a drug tube defining a distal portion of the drug lumen, the drug tube having a distal portion connected to an exterior of the balloon and comprising a plurality of holes configured to allow the drug to exit the drug tube.

### BACKGROUND

The present application relates generally to drug eluting apparatuses for delivering a drug, such as a drug that includes an anti-proliferative agent, into a blood vessel. More specifically, the present application relates to drug eluting apparatuses that allow for delivery of a drug through a drug tube, so as to deliver the drug into the vessel without the drug passing through a balloon.

Vascular diseases may result in stenosis and restenosis of vessels, in which the buildup of plaque on the walls of the vessels results in a narrowing of the vessels, and are detrimental to health if the vessel is not treated. For instance, the vessel may be treated by delivering a drug, such as a drug that includes an anti-proliferative agent. In known methods, a drug eluting apparatus including a balloon may be inserted into a vessel and advanced to the site of the vessel narrowing, and the drug may be delivered to a chamber in the balloon and then out of the balloon via openings in the balloon wall and into the vessel.

### SUMMARY

However, when a drug is delivered via openings in a balloon wall, a significant amount of the drug may remain in the balloon after delivery, due to the relatively large volume of the balloon. Embodiments of the present disclosure may be used to address this issue.

In some embodiments, a drug eluting apparatus includes a handle including a balloon inflation port configured to receive an inflation fluid and a drug port configured to receive a drug. The drug eluting apparatus also includes a shaft connected to the handle. The shaft includes a balloon inflation lumen configured to receive the inflation fluid from the balloon inflation port, a drug lumen configured to receive the drug from the drug port, and a guidewire lumen configured to receive a guidewire and allow the guidewire to extend out of a distal portion of the shaft. The drug eluting apparatus also includes a balloon attached to the distal portion of the shaft and configured to receive the inflation fluid from the balloon inflation lumen. The drug eluting apparatus also includes a drug tube defining a distal portion of the drug lumen, the drug tube having a distal portion connected to an exterior of the balloon and including a plurality of holes configured to allow the drug to exit the drug tube.

In some embodiments, a drug eluting apparatus includes a handle including a drug port configured to receive a drug. The drug eluting apparatus also includes a carrier tube connected to or connectable to the handle, the carrier tube including a lumen. The drug eluting apparatus also includes a drug tube configured to move through the lumen of the carrier tube. The drug tube includes a proximal portion configured to receive a drug from the drug port and a distal portion including a plurality of holes configured to allow the drug to exit the drug tube. When the distal portion of the drug tube is located in the carrier tube, the distal portion of the drug tube is held by the carrier tube in a deformed radially collapsed state. When the distal portion of the drug tube is outside the carrier tube, the distal portion of the drug tube takes on a relaxed radially expanded state.

In some embodiments, a method of delivering a drug into a vessel includes providing a drug eluting apparatus. The drug eluting apparatus includes a handle including a balloon inflation port configured to receive an inflation fluid and a drug port configured to receive a drug. The drug eluting apparatus also includes a shaft connected to the handle. The shaft includes a balloon inflation lumen configured to receive the inflation fluid from the balloon inflation port, a drug lumen configured to receive the drug from the drug port, and a guidewire lumen configured to receive a guidewire and allow the guidewire to extend out of a distal portion of the shaft. The drug eluting apparatus also includes a balloon attached to the distal portion of the shaft and configured to receive the inflation fluid from the balloon inflation lumen. The drug eluting apparatus also includes a drug tube defining a distal portion of the drug lumen, the drug tube having a distal portion connected to an exterior of the balloon and including a plurality of holes configured to allow the drug to exit the drug tube. The method also includes inserting the distal portion of the shaft into a vessel. The method also includes inflating the balloon by providing the inflation fluid to the balloon via the balloon inflation tube, which causes the distal portion of the drug tube to take a deformed radially expanded state. The method also includes delivering the drug into the vessel via the drug tube and the plurality of holes of the drug tube.

In some embodiments, a method of delivering a drug into a vessel includes providing a drug eluting apparatus. The drug eluting apparatus includes a handle including a balloon inflation port configured to receive an inflation fluid and a drug port configured to receive a drug. The drug eluting apparatus also includes a shaft connected to the handle. The shaft includes a balloon inflation lumen configured to receive the inflation fluid from the balloon inflation port, a drug lumen configured to receive the drug from the drug port, and a guidewire lumen configured to receive a guidewire and allow the guidewire to extend out of a distal portion of the shaft. The drug eluting apparatus also includes a balloon attached to the distal portion of the shaft and configured to receive the inflation fluid from the balloon inflation lumen. The drug eluting apparatus also includes a carrier tube connected to the handle, the carrier tube being configured to allow the shaft to move therethrough. The carrier tube has a length that is greater than or equal to a length of the shaft. The drug eluting apparatus also includes a drug tube defining a distal portion of the drug lumen, the drug tube having a distal portion connected to an exterior of the balloon and including a plurality of holes configured to allow the drug to exit the drug tube. The method also includes inserting the distal portion of the shaft into a vessel. The method also includes pushing the shaft or pulling the carrier tube so that the distal portion of the drug tube and the balloon are outside of the carrier tube, which causes the distal portion of the drug tube to take a relaxed radially expanded state. The method also includes inflating the balloon by providing the inflation fluid to the balloon via the balloon inflation tube. The method also includes delivering the drug into the vessel via the drug tube and the plurality of holes of the drug tube.

In some embodiments, a method of delivering a drug into a vessel includes providing a drug eluting apparatus. The drug eluting apparatus includes a handle including a balloon inflation port configured to receive an inflation fluid and a drug port configured to receive a drug. The drug eluting apparatus also includes a shaft connected to the handle. The drug eluting apparatus also includes a shaft connected to the handle. The shaft includes a balloon inflation lumen configured to receive the inflation fluid from the balloon inflation port, a drug lumen configured to receive the drug from the drug port, and a guidewire lumen configured to receive a guidewire and allow the guidewire to extend out of a distal portion of the shaft. The drug eluting apparatus also includes a balloon attached to the distal portion of the shaft and configured to receive the inflation fluid from the balloon inflation lumen. The drug eluting apparatus also includes a carrier tube connected to or connectable to the handle, the carrier tube configured to allow the shaft to move therethrough. The carrier tube is configured to be attached to a guiding catheter. The drug eluting apparatus also includes a drug tube defining a distal portion of the drug lumen, the drug tube having a distal portion connected to an exterior of the balloon and including a plurality of holes configured to allow the drug to exit the drug tube. The carrier tube has a length that is approximately equal to an axial length of the distal portion of the drug tube. The method also includes inserting the distal portion of the shaft into a vessel. The method also includes pushing the shaft so that the distal portion of the drug tube and the balloon are outside of the carrier tube and the guiding catheter, which causes the distal portion of the drug tube to take a relaxed radially expanded state. The method also includes inflating the balloon by providing the inflation fluid to the balloon via the balloon inflation tube. The method also includes delivering the drug into the vessel via the drug tube and the plurality of holes of the drug tube.

In some embodiments, a method of delivering a drug into a vessel includes providing a drug eluting apparatus. The drug eluting apparatus includes a handle including a drug port configured to receive a drug. The drug eluting apparatus also includes a carrier tube connected to the handle, the carrier tube including a lumen. The drug eluting apparatus also includes a drug tube configured to move through the lumen of the carrier tube. The drug tube includes a proximal portion configured to receive a drug from the drug port and a distal portion including a plurality of holes configured to allow the drug to exit the drug tube. When the distal portion of the drug tube is located in the carrier tube, the distal portion of the drug tube is held by the carrier tube in a deformed radially collapsed state. When the distal portion of the drug tube is outside the carrier tube, the distal portion of the drug tube takes on a relaxed radially expanded state. The carrier tube has a length that is equal to or greater than an axial length of the drug tube. The method also includes inserting the distal portion of the carrier tube into a vessel. The method also includes pushing the drug tube or pulling the carrier tube so that the distal portion of the drug tube is outside of the carrier tube, which causes the distal portion of the drug tube to take a relaxed radially expanded state, the drug tube receiving the drug from the drug port. The method also includes delivering the drug into the vessel via the drug tube and the plurality of holes of the drug tube.

In some embodiments, a method of delivering a drug into a vessel includes providing a drug eluting apparatus. The drug eluting apparatus includes a handle including a drug port configured to receive a drug. The drug eluting apparatus also includes a carrier tube connected to or connectable to the handle, the carrier tube including a lumen. The carrier tube is configured to be attached to a guiding catheter. The drug eluting apparatus also includes a drug tube configured to move through the lumen of the carrier tube. The drug tube includes a proximal portion configured to receive a drug from the drug port and a distal portion including a plurality of holes configured to allow the drug to exit the drug tube. When the distal portion of the drug tube is located in the carrier tube, the distal portion of the drug tube is held by the carrier tube in a deformed radially collapsed state. When the distal portion of the drug tube is outside the carrier tube, the distal portion of the drug tube takes on a relaxed radially expanded state. The carrier tube has a length that is approximately equal to an axial length of the distal portion of the drug tube. The method also includes inserting the distal portion of the carrier tube into a vessel. The method also includes pushing the drug tube so that the distal portion of the drug tube is outside of the carrier tube and the guiding catheter, which causes the distal portion of the drug tube to take a relaxed radially expanded state, the drug tube receiving the drug from the drug port. The method also includes delivering the drug into the vessel via the drug tube and the plurality of holes of the drug tube.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a left side view of a drug eluting apparatus according to a first embodiment, with a distal portion of the drug tube taking on a radially expanded state.

FIG. 2A is a left side view of a portion of the drug eluting apparatus according to the first embodiment; FIG. 2B a left side view of a portion of the drug eluting apparatus according to the first embodiment.

FIG. 3A is a cross-sectional view of the portion of the drug eluting apparatus according to the first embodiment taken along the plane A-A in FIG. 2A; FIG. 3B is a cross-sectional view of the portion of the drug eluting apparatus according to the first embodiment taken along the plane B-B in FIG. 2A; and FIG. 3C is a cross-sectional view of the portion of the drug eluting apparatus according to the first embodiment taken along the plane C-C in FIG. 2A.

FIG. 4A is a cross-sectional view of the portion of the drug eluting apparatus according to the first embodiment taken along the plane A-A in FIG. 2A; FIG. 4B is a cross-sectional view of the portion of the drug eluting apparatus according to the first embodiment taken along the plane B-B in FIG. 2A; and FIG. 4C is a cross-sectional view of the portion of the drug eluting apparatus according to the first embodiment taken along the plane C-C in FIG. 2A.

FIG. 5A is a cross-sectional view of the portion of the drug eluting apparatus according to the first embodiment taken along the plane A-A in FIG. 2A; FIG. 5B is a cross-sectional view of the portion of the drug eluting apparatus according to the first embodiment taken along the plane B-B in FIG. 2A; and FIG. 5C is a cross-sectional view of the portion of the drug eluting apparatus according to the first embodiment taken along the plane C-C in FIG. 2A.

FIG. 6A is a cross-sectional view of the portion of the drug eluting apparatus according to the first embodiment taken along the plane A-A in FIG. 2A; FIG. 6B is a cross-sectional view of the portion of the drug eluting apparatus according to the first embodiment taken along the plane B-B in FIG. 2A; and FIG. 6C is a cross-sectional view of the portion of the drug eluting apparatus according to the first embodiment taken along the plane C-C in FIG. 2A.

FIG. 7A is a cross-sectional view of the portion of the drug eluting apparatus according to the first embodiment taken along the plane A-A in FIG. 2A; FIG. 7B is a cross-sectional view of the portion of the drug eluting apparatus according to the first embodiment taken along the plane B-B in FIG. 2A; and FIG. 7C is a cross-sectional view of the portion of the drug eluting apparatus according to the first embodiment taken along the plane C-C in FIG. 2A.

FIG. 8 is a left side view of a portion of the drug eluting apparatus of the first embodiment, with the distal portion of the drug tube extending in a helical shape.

FIG. 9A is a left side view of a drug eluting apparatus according to a first embodiment, with the distal portion of the drug tube having a plurality of branching drug; FIG. 9B is a view of a portion of the distal portion of the drug tube of the first embodiment in FIG. 9A shown by the arrow X in FIG. 9A, with the distal portion of the drug tube having a lattice shape.

FIG. 10A is a left side view of a drug eluting apparatus according to a second embodiment, with a distal portion of the drug tube in a relaxed radially expanded state and extending in a helical shape; FIG. 10B is a left side view of the drug tube of the second embodiment in FIG. 10A.

FIG. 11A is a left side view of a portion of the drug eluting apparatus according to a second embodiment, with the distal portion of the drug tube in a relaxed radially expanded state and having a plurality of drug tube portions that extend from a proximal end of the distal portion to a distal end of the distal portion and are curved radially outward relative to a longitudinal axis of a carrier tube; FIG. 11B is a left side view of the drug tube of the second embodiment in FIG. 11A.

FIG. 12A is a right side view of a drug eluting apparatus for use with a short carrier tube and a guiding catheter, in a first configuration; FIG. 12B is a right side view of the drug eluting apparatus for use with the short carrier tube and the guiding catheter, in a second configuration; FIG. 12C is a right side view of the drug eluting apparatus for use with the short carrier tube and the guiding catheter, in a third configuration; and FIG. 12D is a right side view of the drug eluting apparatus for use with the short carrier tube and the guiding catheter, in a fourth configuration.

FIG. 13A is a right side view of a drug eluting apparatus for use with a long carrier tube, in a first configuration; FIG. 13B is a right side view of the drug eluting apparatus for use with the long carrier tube, in a second configuration; FIG. 13C is a right side view of the drug eluting apparatus for use with the long carrier tube, in a third configuration.

FIG. 14A is a right side view of a distal portion of a drug eluting apparatus for use with an inserter, a valve, a guiding catheter, and a flushing device in a first configuration; FIG. 14B is a right side view of the distal portion of the drug eluting apparatus for use with the inserter, the valve, the guiding catheter, and the flushing device, in a second configuration; FIG. 14C is a right side view of the distal portion of the drug eluting apparatus for use with the inserter, the valve, the guiding catheter, and the flushing device, in a third configuration; and FIG. 14D is a right side view of the distal portion of the drug eluting apparatus for use with the inserter, the valve, the guiding catheter, and the flushing device, in a fourth configuration.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

To deliver a drug into a vessel using a drug eluting apparatus, it may be desirable to deliver the drug through a structure other than a balloon, that is, other than directly through openings in a balloon wall.

Various embodiments of the drug eluting apparatuses described herein may provide one or more advantages, including, for example: (1) allowing for an inflation fluid for the balloon to be different from the drug that is delivered into the vessel, so that there is less waste of the drug that would otherwise remain in the balloon after the drug is delivered to the vessel; and (2) allowing for delivery of the drug into the vessel via a drug eluting apparatus without a balloon.

Various embodiments of the drug eluting apparatuses described herein may be used with a guiding catheter. A guiding catheter may be inserted into a vessel before the drug eluting apparatuses are inserted. The drug eluting apparatuses may be connected to the guiding catheter to facilitate entry of portions of the drug eluting apparatuses into the vessel. Use of a guiding catheter may protect the vessel from potential trauma that could be caused by the use of the drug eluting apparatuses without a guiding catheter. The guiding catheter may be positioned at or near the target of drug elution.

Various embodiments of the drug eluting apparatuses described herein may be used with a carrier tube. A carrier tube may surround a drug tube of and/or the balloon of the drug eluting apparatuses. The carrier tube may facilitate entry of portions of the drug eluting apparatuses into the vessel. Use of a carrier tube may provide an external force on a distal portion of the drug tube to hold the distal portion of the drug tube in a deformed radially compressed state. The carrier tube may be positioned at or near the target of drug elution.

### First Embodiment

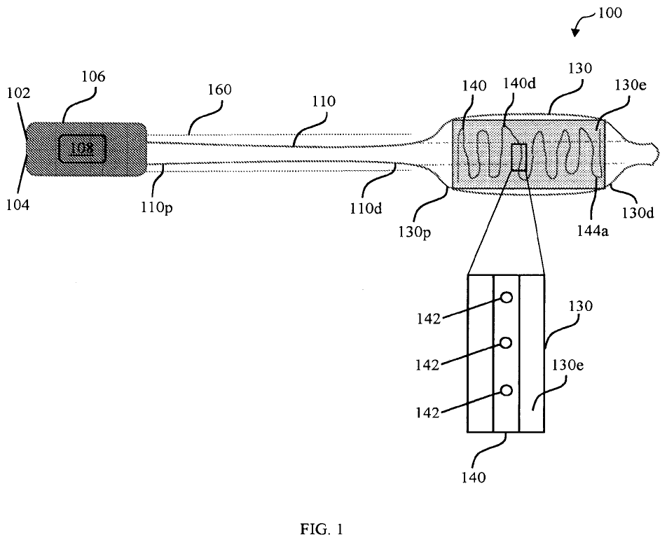

FIG. 1-9B depict a drug eluting apparatus 100 (e.g., treatment apparatus, etc.) for delivering a drug (e.g., a drug that includes an anti-proliferative agent such as sirolimus, etc.) into a vessel according to a first embodiment. The drug eluting apparatus 100 includes a handle 106 including a balloon inflation port 102 configured to receive an inflation fluid and a drug port 104 configured to receive a drug. The drug eluting apparatus 100 also includes a shaft 110 connected to the handle 106. The shaft 110 includes a balloon inflation lumen 113 configured to receive the inflation fluid from the balloon inflation port 102, a drug lumen 114 configured to receive the drug from the drug port 104, and a guidewire lumen 117 configured to receive a guidewire 126 and allow the guidewire 126 to extend out of a distal portion 110d of the shaft 110. The drug eluting apparatus 100 also includes a balloon 130 attached to the distal portion 110d of the shaft 110 and configured to receive the inflation fluid from the balloon inflation tube 112. The drug eluting apparatus 100 also includes a drug tube 140 defining a distal portion 114d of the drug lumen 114, the drug tube 140 having a distal portion 140d connected to an exterior 130e of the balloon 130 and including a plurality of holes 142 configured to allow the drug to exit the drug tube 140. In some embodiments, the drug eluting apparatus 100 may further include a carrier tube 160 connected to or connectable to the handle 106. The carrier tube 160 is configured to allow the shaft 110 to move therethrough. In some embodiments, the handle 106 comprises an actuator 108, the actuator 108 configured to move the shaft 110 relative to the carrier tube 160 or to move the carrier tube 160 relative to the shaft 110. In some embodiments, the drug eluting apparatus 100 may further include a guidewire tube 116 in which the guidewire lumen 117 is formed. In some embodiments, the drug eluting apparatus 100 may further include a balloon inflation tube 112 in which the balloon inflation lumen 113 is formed.

The drug eluting apparatus 100 includes a handle 106 (e.g., grip, handgrip, etc.). The handle 106 is configured to be held by a user and allows a user to operate the drug eluting apparatus 100. The handle 106 may be configured to be held with one hand, so as to allow a user to perform additional maneuvers with the user's second hand.

The handle 106 may be made of polyethylene, polyolefins (e.g., polypropylene, polyamides), polyesters (e.g., polyethylene terephthalate), fluoropolymers (such as polytetrafluoroethylene (PTFE)), ethylene/tetrafluoroethylene copolymer (ETFE), polyetheretherketone (PEEK), polyamides, elastomer, polyester elastomer, polycarbonate, polyetherimide, polystyrene, polyacetal, ABS (acrylonitrile, butadiene, styrene copolymer synthetic) resin, or other suitable materials.

The handle 106 also includes a balloon inflation port 102 (e.g., hole, opening, aperture, etc.) configured to receive an inflation fluid. The inflation fluid may be saline, contrast, a mixture of saline and contrast, or other suitable inflating fluids. In FIG. 1, the balloon inflation port 102 is located at a proximal side of the handle 106. However, in other embodiments, the balloon inflation port 102 may be located at the top, bottom, left, right, or proximal side of the handle 106. In yet other embodiments, the balloon inflation port 102 may be located on a proximal portion 110p of the shaft 110.

The handle 106 also includes a drug port 104 (e.g., hole, opening, aperture, etc.) configured to receive a drug. In FIG. 1, the drug port 104 is located at a proximal side of the handle 106. However, in other embodiments, the drug port 104 may be located at the top, bottom, left, right, or proximal side of the handle 106. In yet other embodiments, the drug port 104 may be located on a proximal portion 110p of the shaft 110.

In some embodiments, the handle 106 includes an actuator 108 (e.g., tab, button, wheel, knob, etc.). The actuator 108 may be a mechanical structure that is actuatable by a user to operate the drug eluting apparatus 100. In FIG. 1, the actuator 108 is located at a top side of the handle 106. However, in other embodiments, the actuator 108 may be located at a bottom, left, or right side of the handle 106. In some embodiments, the actuator 108 is configured to move the shaft 110 relative to the carrier tube 160. The shaft 110 may be rotated in a clockwise or a counterclockwise direction about the longitudinal axis A_{L} and/or may be moved in a longitudinal direction along the longitudinal axis A_{L}. In other embodiments, the actuator 108 is configured to move the carrier tube 160 relative to the shaft 110. The carrier tube 160 may be rotated in a clockwise or a counterclockwise direction about the longitudinal axis A_{L} and/or may be moved in a longitudinal direction along the longitudinal axis A_{L}.

The drug eluting apparatus 100 also includes a shaft 110 (e.g., catheter, tube, pipe, cylinder, conduit, etc.). The shaft 110 is connected to the handle 106. The shaft 110 may be connected to the handle 106 by bonding with an adhesive, by an interlocking feature, by press fit, by threaded fastener, or by other suitable connecting arrangements. The shaft may be permanently fixed to the handle 106 so that the user may not disconnect the shaft 110 from the handle 106. The shaft 110 may have a cap 111 at the distal portion 110d of the shaft 110.

The shaft 110 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, or other suitable materials.

The shaft 110 may have a length that is approximately in a range of 30-300 cm, for example, 50-300 cm, or 100-250 cm. As used herein, a range of X to Y includes X, Y, and values between X and Y.

The shaft 110 may have a cross-sectional shape that is a circle, an oval, a polygon, a rounded polygon, or other geometric shape. The shaft 110 may have a maximum cross-sectional width (e.g., a diameter where the cross-sectional shape is circular) that is approximately in a range of 0.5-10 mm, inclusive, and ideally, 0.5-7 mm.

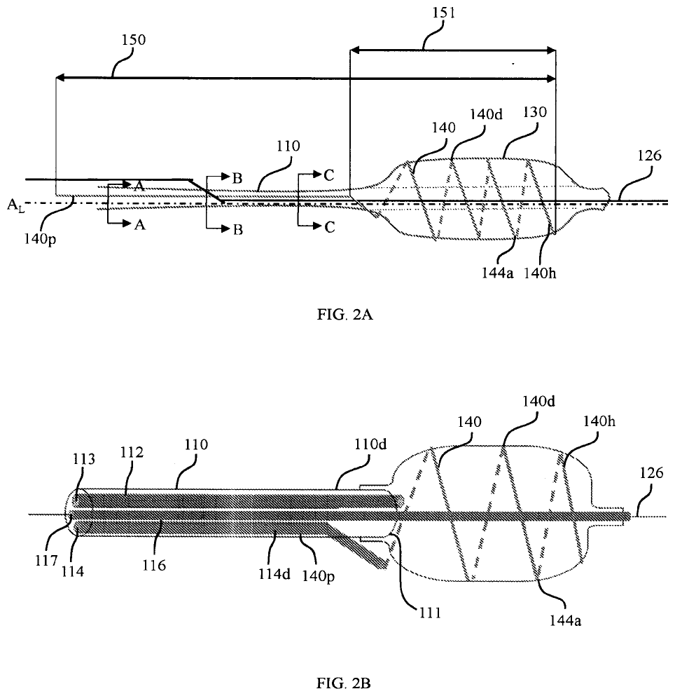

FIG. 2A and 2B show a portion of the drug eluting apparatus 100. The drug eluting apparatus 100 may be a rapid-exchange type device, in which the guidewire 126 enters the shaft 110 at a location of the shaft 110 that is distal to the location where the shaft 110 is connected to the handle 106. In other embodiments, the drug eluting apparatus 100 may be an over-the-wire type device, in which the guidewire 126 enters the shaft 110 through the handle 106.

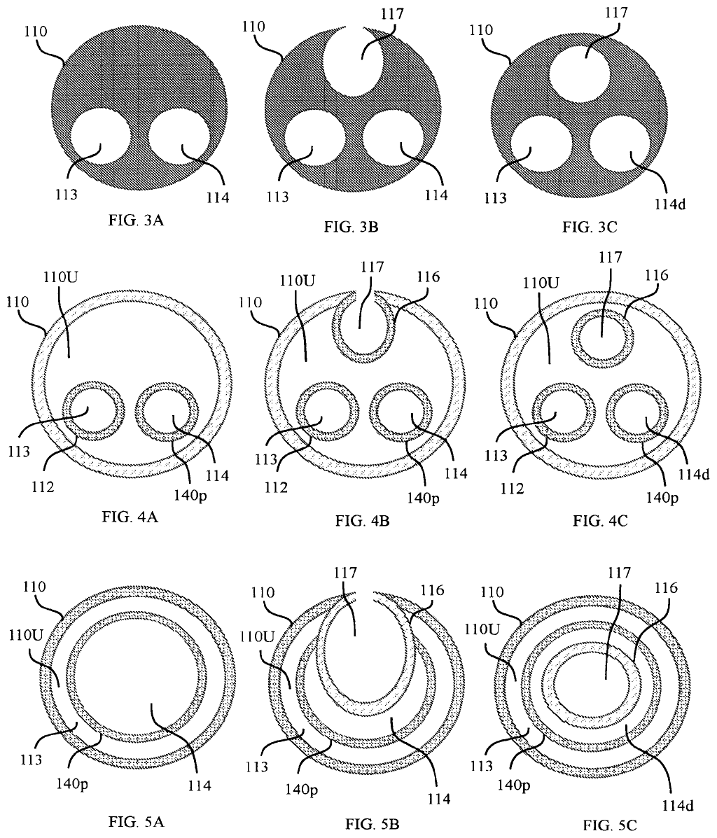

In FIG. 3A-3C, the shaft 110 is solid. The shaft 110 includes a balloon inflation lumen 113 (e.g., cavity, space, etc.), a drug lumen 114 (e.g., cavity, space, etc.), and a guidewire lumen 117 (e.g., pipe, cylinder, conduit, etc.). FIG. 3A-3C show cross-sections of the solid shaft 110 at planes A-A, B-B, and C-C respectively of FIG. 2A. In the embodiment of FIG. 3A-3C, the balloon inflation lumen 113, the drug lumen 114, and the guidewire lumen 117 are separate from each other.

The balloon inflation lumen 113 extends longitudinally through the shaft 110. The balloon inflation lumen 113 is configured to receive the drug from the balloon inflation port 102. The balloon inflation lumen 113 may have a cross-sectional shape that is a circle, an oval, a polygon, a rounded polygon, or other geometric shape. The balloon inflation lumen 113 may have a maximum cross-sectional width (e.g., a diameter where the cross-sectional shape is circular) that is approximately in a range of 1 μm to 15 mm, inclusive, and for example 0.1-3 mm.

The drug lumen 114 extends longitudinally through the shaft 110. The drug lumen 114 is configured to receive the drug from the drug port 104. The drug lumen 114 may have a cross-sectional shape that is a circle, an oval, a polygon, a rounded polygon, or other geometric shape. The drug lumen 114 may have a maximum cross-sectional width (e.g., a diameter where the cross-sectional shape is circular) that is approximately in a range of 1 μm to 15 mm, inclusive, and ideally 0.1-3 mm.

The guidewire lumen 117 extends longitudinally through the shaft 110. The guidewire lumen 117 is configured to receive the guidewire 126. The guidewire lumen 117 may have a cross-sectional shape that is a circle, an oval, a polygon, a rounded polygon, or other geometric shape. The guidewire lumen 117 may have a maximum cross-sectional width (e.g., a diameter where the cross-sectional shape is circular) that is approximately in a range of 1-15 mm, inclusive, and for example 1-3 mm.

In FIG. 3A-3C, the balloon inflation lumen 113, the drug lumen 114, and the guidewire lumen 117 are positioned in the shaft 110 such that the balloon inflation lumen 113 is at the bottom-left of the shaft 110, the drug lumen 114 is at the bottom-right of the shaft 110, and the guidewire lumen 117 is at the top of the shaft 110. In other embodiments, the balloon inflation lumen 113, the drug lumen 114, and the guidewire lumen 117 may be positioned at other locations of the shaft 110 and with different relative positioning to each other, as compared to the arrangement of FIG. 3A-3C.

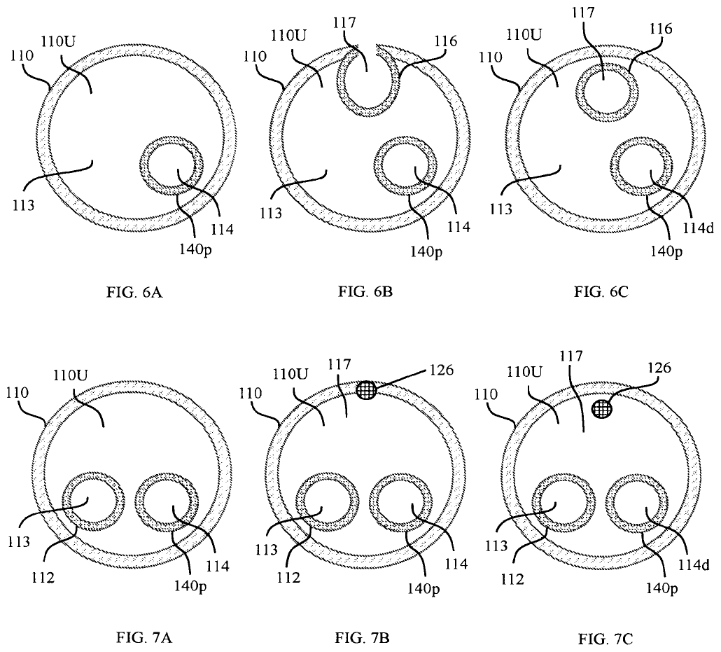

In FIG. 4A-7C the shaft 110 is hollow and has a shaft lumen 110U (e.g., cavity, space, etc.). The shaft 110 may include a balloon inflation tube 112 (e.g., pipe, cylinder, conduit, etc.), a proximal portion 140p of a drug tube 140 (e.g., pipe, cylinder, conduit, etc.), and/or a guidewire tube 116 (e.g., pipe, cylinder, conduit, etc.). FIG. 4A-4C show cross-sections of the shaft 110 at planes A-A, B-B, and C-C respectively of FIG. 2A. In the embodiment of FIG. 4A-4C, the guidewire tube 116 is separate from the proximal portion 140p of the drug tube 140. FIG. 5A-5C show cross-sections of the shaft 110 at planes A-A, B-B, and C-C respectively of FIG. 2A. In the embodiment of FIG. 5A-5C, the shaft 110 does not have a separate balloon inflation tube 112, the shaft lumen 110U of the shaft 110 is used as the balloon inflation lumen 113 to receive the inflation fluid, and the guidewire tube 116 extends through the proximal portion 140p of the drug tube 140. FIG. 6A-6C show cross-sections of the shaft 110 at planes A-A, B-B, and C-C respectively of FIG. 2A. In the embodiment of FIG. 6A-6C, the shaft 110 does not have a separate balloon inflation tube 112, and the shaft lumen 110U of the shaft 110 is used as the balloon inflation lumen 113 to receive the inflation fluid. FIG. 7A-7C show cross-sections of the shaft 110 at planes A-A, B-B, and C-C respectively of FIG. 2A. In the embodiment of FIG. 7A-7C, the shaft 110 does not have a separate guidewire tube 116, and the shaft lumen 110U of the shaft 110 is used as the guidewire lumen 117 to receive the guidewire 126 directly.

The balloon inflation tube 112 extends longitudinally through the shaft 110. The balloon inflation lumen 113 is formed in the balloon inflation tube 112. The balloon inflation tube 112 may have a cross-sectional shape that is a circle, an oval, a polygon, a rounded polygon, or other geometric shape. The balloon inflation tube 112 may have a maximum cross-sectional width (e.g., a diameter where the cross-sectional shape is circular) that is approximately in a range of 0.1-1 mm, inclusive, and for example 0.3-0.9 mm.

The balloon inflation tube 112 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, or other suitable materials.

The proximal portion 140p of the drug tube 140 extends longitudinally through the shaft 110. The drug lumen 114 is formed in the proximal portion 140p of the drug tube 140. The proximal portion 140p of the drug tube 140 may have a cross-sectional shape that is a circle, an oval, a polygon, a rounded polygon, or other geometric shape. The proximal portion 140p of the drug tube 140 may have a maximum cross-sectional width (e.g., a diameter where the cross-sectional shape is circular) that is approximately in a range of 0.1-1 mm, inclusive, and ideally 0.3-0.9 mm.

The guidewire tube 116 is configured to receive a guidewire 126 and allow the guidewire 126 to extend out of a distal portion 110d of the shaft 110, as in FIG. 2A and 2B. The guidewire lumen 117 is formed in the guidewire tube 116. The guidewire tube 116 extends longitudinally through a portion of the shaft 110 and through the balloon 130. The guidewire tube 116 may have a cross-sectional shape that is a circle, an oval, a polygon, a rounded polygon, or other geometric shape. The guidewire tube 116 may have a maximum cross-sectional width (e.g., a diameter where the cross-sectional shape is circular) that is approximately in a range of 0.1-1 mm, inclusive, and ideally 0.3-0.9 mm.

The guidewire tube 116 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, or other suitable materials.

In FIG. 4A-4C, the balloon inflation tube 112, the proximal portion 140p of the drug tube 140, and the guidewire tube 116 are positioned in the shaft 110 such that the balloon inflation tube 112 is at the bottom-left of the shaft 110, the proximal portion 140p of the drug tube 140 is at the bottom-right of the shaft 110, and the guidewire tube 116 is at the top of the shaft 110. In other embodiments, the balloon inflation tube 112, the proximal portion 140p of the drug tube 140, and the guidewire tube 116 may be positioned at other locations of the shaft 110 and with different relative positioning to each other, as compared to the arrangement of FIG. 4A-4C.

In FIG. 6A-6C, the proximal portion 140p of the drug tube 140 and the guidewire tube 116 are positioned in the shaft 110 such that the proximal portion 140p of the drug tube 140 is at the bottom-right of the shaft 110, and the guidewire tube 116 is at the top of the shaft 110. In other embodiments, the proximal portion 140p of the drug tube 140 and the guidewire tube 116 may be positioned at other locations of the shaft 110 and with different relative positioning to each other, as compared to the arrangement of FIG. 6A-6C.

In FIG. 7A-7C, the balloon inflation tube 112, the proximal portion 140p of the drug tube 140, and the guidewire 126 are positioned in the shaft 110 such that the balloon inflation tube 112 is at the bottom-left of the shaft 110, the proximal portion 140p of the drug tube 140 is at the bottom-right of the shaft 110, and the guidewire 126 is at the top of the shaft 110. In other embodiments, the balloon inflation tube 112, the proximal portion 140p of the drug tube 140, and the guidewire 126 may be positioned at other locations of the shaft 110 and with different relative positioning to each other, as compared to the arrangement of FIG. 7A-7C.

In embodiments in which the shaft 110 is hollow and has a shaft lumen 110U, the shaft 110 has the cap 111 at the distal portion 110d of the shaft 110, as in FIG. 2B. The cap 111 at the distal portion 110d of the shaft 110 may extend around the balloon inflation tube 112 and the guidewire tube 116 such that the balloon inflation tube 112 and the guidewire tube 116 pass through the cap 111 of the shaft 110. The cap 111 of the shaft 110 prevents inflation fluid that has entered the balloon 130 from the balloon inflation tube 112 from entering the shaft lumen 110U of the shaft 110 from the balloon 130, as in FIG. 2B.

The guidewire tube 116 extends through the balloon 130 and out of the balloon 130 through a hole at the distal end 130d of the balloon 130. The distal end 130d of the balloon 130 may be connected to the outside of the guidewire tube 116, as in FIG. 2B, by bonding with an adhesive, or by other suitable arrangements.

The drug eluting apparatus 100 also includes a balloon 130. The balloon 130 is attached to the distal portion 110d of the shaft 110. The balloon 130 may be attached to the distal portion 110d of the shaft 110 by being placed over the distal portion 110d of the shaft 110 and bonded with an adhesive, or by other suitable connecting arrangements. The balloon 130 is configured to receive the inflation fluid from the balloon inflation tube 112. When the inflation fluid enters the balloon 130 from the balloon inflation tube 112, the balloon 130 is inflated.

The balloon 130 may be made of polyamide, polyester, polyurethane, polyamide elastomer, polyester elastomer, polyurethane elastomer, or other suitable material.

The balloon 130 may have a length that is approximately in a range of 10-400 mm. The balloon 130 may have a width that is approximately in a range of 1-8 mm.

The drug eluting apparatus 100 also includes a drug tube 140 (e.g., pipe, cylinder, conduit, etc.). The drug tube 140 defines a distal portion 114d of the drug lumen 114. The drug tube 140 is configured to receive the drug from the drug lumen 114. In embodiments with a hollow shaft 110 having a shaft lumen 110U, the drug tube 140 has a proximal portion 140p that extends longitudinally through the shaft 110, the drug lumen 114 is formed in the proximal portion 140p of the drug tube 140, and the drug tube 140 has a distal portion 140d that is outside of the shaft 110 at a distal portion 110d of the shaft 110. The distal portion 140d of the drug tube 140 is connected to the exterior 130e of the balloon 130. In embodiments with a solid shaft 110, the drug lumen 114 extends longitudinally through the shaft 110, the drug tube 140 defines a distal portion 114d of the drug lumen 114, the drug tube 140 is outside of the shaft 110 at a distal portion 110d of the shaft 110, and the drug tube 140 is connected to the exterior 130e of the balloon 130. The drug tube 140 of the embodiments with the solid shaft 110 is equivalent to the distal portion 140d of the drug tube 140 of the embodiments with the hollow shaft 110 having a shaft lumen 110U.

At least the distal portion 140d of the drug tube 140 may be made of an elastically deformable material. In some embodiments, the drug tube 140 may be made of nitinol or stainless steel.

In embodiments with a hollow shaft 110 having a shaft lumen 110U, in which the drug tube 140 has a proximal portion 140p that extends longitudinally through the shaft 110 and a distal portion 140d that is outside of the shaft 110, the drug tube 140 may have an axial length 150 that is approximately in a range of 30 to 330 cm, for example, 50-330 cm, or 100-250 cm. The axial length 150 of the drug tube 140 is measured along the longitudinal axis A_{L} as in FIG. 2A. The distal portion 140d of the drug tube 140 may have an axial length 151 that is approximately in a range of 30 to 300 mm, for example, 50-300 mm, or 100-250 mm. The axial length 151 of the distal portion 140d of the drug tube 140 is measured along the longitudinal axis A_{L} as in FIG. 2A.

In embodiments with a solid shaft 110, in which the drug tube 140 is outside of the shaft 110, the drug tube 140 may have an axial length that is 30 to 300 mm, for example, 50-300 mm, or 100-250 mm (similar to the axial length 151 of the distal portion of the drug tube 140 in the embodiment with the hollow shaft 110). The axial length of the drug tube 140 is measured along the longitudinal axis A_{L} as in FIG. 2A.

The drug tube 140 may have a cross-sectional shape that is a circle, an oval, a polygon, a rounded polygon, or other geometric shape. The drug tube 140 may have a maximum cross-sectional width (e.g., a diameter where the cross-sectional shape is circular) that is approximately in a range of 0.1-1 mm, inclusive, and ideally 0.3-0.9 mm.

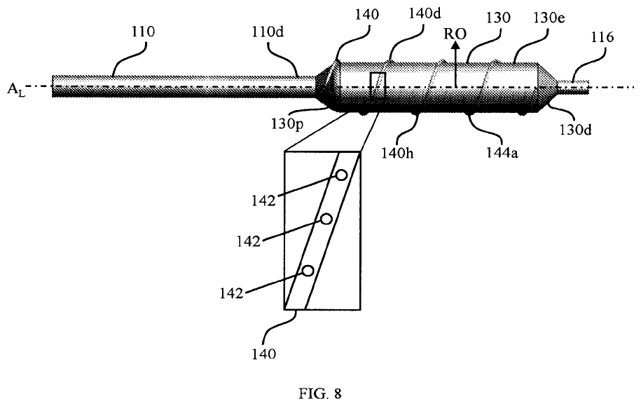

The distal portion 140d of the drug tube 140 includes a plurality of holes 142 (e.g., apertures, openings, slits, etc.) that are configured to allow the drug to exit the distal portion 140d of the drug tube 140. When the distal portion 140d of the drug tube 140 takes on a radially expanded state 144a, which may be the deformed radially expanded state 144a or the relaxed radially expanded state 144a, the drug passes through the plurality of holes 142 of the distal portion 140d of the drug tube 140 to exit the drug tube 140 and enter the vessel. In some embodiments, the plurality of holes 142 are positioned on the drug tube 140 in a radially outward direction RO relative to a longitudinal axis A_{L} of the shaft 110, as shown in FIG. 8.

The plurality of holes 142 of the distal portion 140d of the drug tube 140 may be manufactured by laser drilling. The plurality of holes 142 of the distal portion 140d of the drug tube 140 may each have a width that is approximately in a range of 100 nm to 10 μm, and ideally, 100 nm to 5 μm. In FIG. 1, the plurality of holes 142 of the distal portion 140d of the drug tube 140 have the same widths. However, in other embodiments, the plurality of holes 142 of the distal portion 140d of the drug tube 140 may have different widths.

The plurality of holes 142 of the distal portion 140d of the drug tube 140 may each have a shape that is a circle, an oval, a rounded polygon, a slit, or other geometric shape. In FIG. 1, the plurality of holes 142 of the distal portion 140d of the drug tube 140 have the same shape. However, in other embodiments, the plurality of holes 142 of the distal portion 140d of the drug tube 140 may have different shapes.

In the embodiment of FIG. 1, the plurality of holes 142 of the distal portion 140d of the drug tube 140 are positioned with a uniform spacing between the plurality of holes 142 of the distal portion 140d of the drug tube 140. However, in other embodiments, the plurality of holes 142 of the distal portion 140d of the drug tube 140 may be positioned with a varying spacing between the plurality of holes 142 of the distal portion 140d of the drug tube 140.

In some embodiments, before the balloon 130 is inflated, the distal portion 140d of the drug tube 140 is in a relaxed radially collapsed state, and when the balloon 130 is inflated with the inflation fluid, the distal portion 140d of the drug tube 140 is expanded into a deformed radially expanded state 144a by the inflation of the balloon 130. In these embodiments, the deformed state of the distal portion 140d of the drug tube 140 is the deformed radially expanded state 144a. The deformed state is the state in which an external force is applied to the distal portion 140d of the drug tube 140. In these embodiments, the relaxed state of the distal portion 140d of the drug tube 140 is the relaxed radially collapsed state. The relaxed state is the state in which no external force is applied to the distal portion 140d of the drug tube 140. When the balloon 130 is inflated, the inflation of the balloon 130 provides an external force that causes the distal portion 140d of the drug tube 140 to enter the deformed radially expanded state 144a. The distal portion 140d of the drug tube 140 changes from the deformed radially expanded state 144a to the relaxed radially collapsed state when the balloon 130 is no longer inflated and so the balloon 130 no longer applies an external force to the distal portion 140d of the drug tube 140.

In some embodiments, the drug eluting apparatus 100 may also include a carrier tube 160 (e.g., a sheath, guiding sheath, cover, etc.) configured to allow the shaft 110 to move therethrough. The carrier tube surrounds the shaft 110 such that the shaft 110 is configured to extend longitudinally through the carrier tube 160. The carrier tube 160 is connected to or connectable to the handle 106. When the carrier tube 160 is connected to the handle 106, the carrier tube 160 is connected to the handle 106 by a Luer lock connector, a lock connector, a fitting connector, or by other suitable connecting arrangements. The carrier tube 160 may be a long carrier tube 180 (discussed in further detail herein), as in FIG. 1. In other embodiments, the carrier tube 160 may be a short carrier tube 182 (discussed in further detail herein). In some embodiments, the carrier tube 160 is configured to be attached to a guiding catheter 184 (discussed in further detail herein).

The carrier tube 160 may be made of fluorine polymers (e.g., polytetrafluoroethylene (PTFE), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, tungsten, stainless steel, Ni—Ti, or other suitable materials.

The carrier tube 160 has a length that is approximately in a range of 30-300 cm, for example, 30-200 cm.

The carrier tube 160 has a cross-sectional shape that is a circle, an oval, a polygon, a rounded polygon, or other geometric shape. The carrier tube 160 may have a maximum cross-sectional width (e.g., a diameter where the cross-sectional shape is circular) that is approximately in a range of 1-15 mm, inclusive, and for example, 1-8 mm. The carrier tube 160 may have a wall having a thickness that is approximately in a range of 0.1-1 mm, inclusive.

In embodiments including the carrier tube 160, when the balloon 130 and the distal portion 140d of the drug tube 140 are located in the carrier tube 160, the distal portion 140d of the drug tube 140 is held by the carrier tube 160 in the deformed radially collapsed state, and when the balloon 130 and distal portion 140d of the drug tube 140 are outside the carrier tube 160, the distal portion 140d of the drug tube 140 takes on the relaxed radially expanded state 144a. In these embodiments, the deformed state of the distal portion 140d of the drug tube 140 is the deformed radially collapsed state, and the distal portion 140d of the drug tube 140 is in the deformed state when in the carrier tube 160. In these embodiments, the relaxed state of the distal portion 140d of the drug tube 140 is the relaxed radially expanded state 144a, and the distal portion 140d of the drug tube 140 may enter the relaxed radially expanded state 144a when the distal portion 140d of the drug tube 140 is outside of the carrier tube 160 and before, simultaneously as, or after the balloon 130 is inflated.

In the embodiment of FIG. 8, the distal portion 140d of the drug tube 140 extends in a helical shape 140h when the distal portion 140d of the drug tube is in the deformed radially expanded state 144a or the relaxed radially expanded state 144a. In a helical shape 140h, the distal portion 140d of the drug tube 140 has a shape that consists of a series of rotations along the distal portion 140d of the drug tube 140 from a proximal end 130p of the balloon 130 to a distal end 130d of the balloon 130.

The rotations of the helical shape 140h of the distal portion 140d of the drug tube 140 may be circular, elliptical, or other rounded arrangements. In FIG. 2A, 2B, and 8, the rotations of the helical shape 140h of the distal portion 140d of the drug tube 140 have the same spacing in between each rotation. However, in other embodiments, the rotations of the helical shape 140h of the distal portion 140d of the drug tube 140 may have varying spacing in between each rotation.

In the embodiment of FIG. 1, the distal portion 140d of the drug tube 140, when in the deformed radially expanded state 144a or the relaxed radially expanded state 144a, snakes circumferentially back and forth in a direction from the proximal end 130p of the balloon 130 to the distal end 130d of the balloon 130. In FIG. 1, the distal portion 140d of the drug tube 140 is shown making twelve turns as the distal portion 140d of the drug tube 140 snakes circumferentially back and forth in a direction from the proximal end 130p of the balloon 130 to the distal end 130d of the balloon 130. However, in other embodiments, the distal portion 140d of the drug tube 140 may make any feasible number of turns as the distal portion 140d of the drug tube 140 snakes circumferentially back and forth in a direction from the proximal end 130p of the balloon 130 to the distal end 130d of the balloon 130.

In the embodiment of FIG. 1, the distal portion 140d of the drug tube 140 is shown snaking circumferentially back and forth in a direction from the proximal end 130p of the balloon 130 to the distal end 130d of the balloon 130 over a portion of the exterior 130e of the balloon 130. However, in other embodiments, the distal portion 140d of the drug tube 140 may snake circumferentially back and forth in a direction from the proximal end 130p of the balloon 130 to the distal end 130d of the balloon 130 over the entirety of the exterior 130e of the balloon

In the embodiment of FIG. 1, the distal portion 140d of the drug tube 140 is shown as having approximately similar axial lengths in between the turns of the distal portion 140d of the drug tube 140 as the distal portion 140d of the drug tube 140 snakes circumferentially back and forth in a direction from the proximal end 130p of the balloon 130 to the distal end 130d of the balloon 130. However, in other embodiments, the distal portion 140d of the drug tube 140 may have varying axial lengths in between the turns of the distal portion 140d of the drug tube 140 as the distal portion 140d of the drug tube 140 snakes circumferentially back and forth in a direction from the proximal end 130p of the balloon 130 to the distal end 130d of the balloon 130.

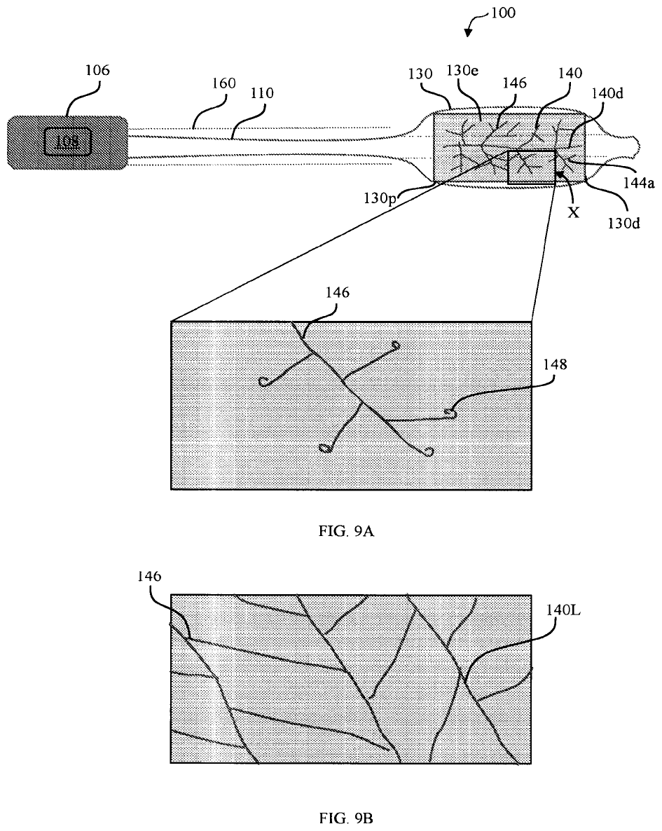

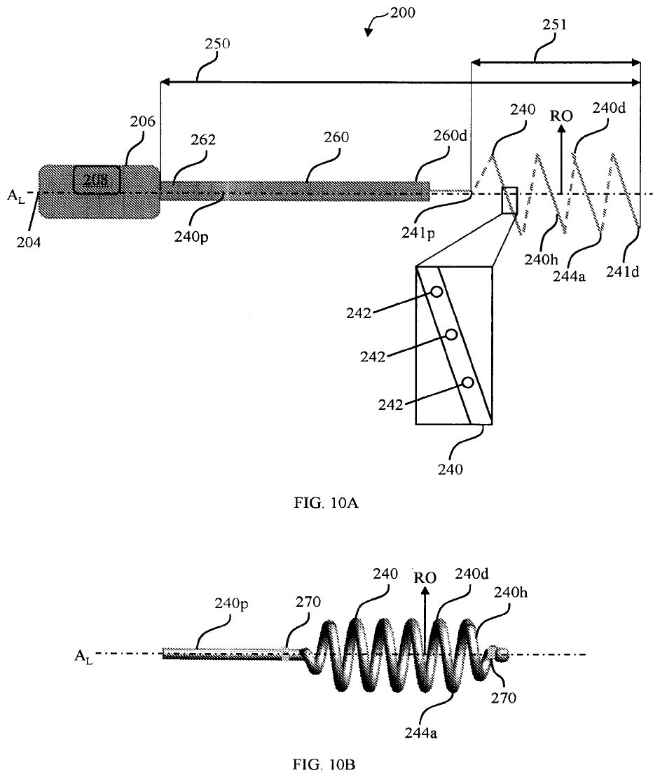

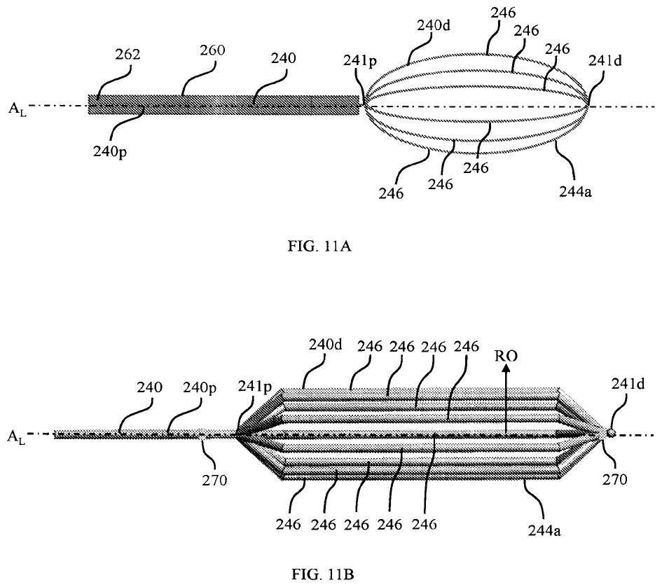

In the embodiments of FIG. 9A and 9B, the distal portion 140d of the drug tube 140 includes a plurality of branching drug tubes 146 that are distributed over the exterior 130e of the balloon 130. The plurality of branching drug tubes 146 may extend in any direction on the exterior 130e of the balloon 130. The plurality of branching drug tubes 146 branch from the distal portion 140d of the drug tube 140, and the plurality of branching drug tubes 146 may also branch from each other. In FIG. 9A, the plurality of branching drug tubes 146 are each shown as having an approximately similar width. However, in other embodiments, the plurality of branching drug tubes 146 may have varying widths. In FIG. 9A, the plurality of branching drug tubes 146 are each shown as having an approximately similar length. However, in other embodiments, the plurality of branching drug tubes 146 may have varying lengths.

In the embodiments of FIG. 9A and 9B, the plurality of branching drug tubes 146 are positioned on a portion of the exterior 130e of the balloon 130. However, in other embodiments, the plurality of branching drug tubes 146 may be positioned over the entirety of the exterior 130e of the balloon 130.

In the embodiment of FIG. 9A, the plurality of branching drug tubes 146 each have an end portion 148 that curls back on itself. Having the end portion 148 that curls back on itself on the plurality of branching drug tubes 146 allows the end portion 148 to be less likely to cause trauma to a vessel as compared to a plurality of branching drug tubes 146 without the end portion 148 that curls back on itself.

In the embodiment of FIG. 9B, the plurality of branching drug tubes 146 have a lattice shape 140L. The plurality of branching drug tubes 146 are connected to each other such that the plurality of branching drug tubes 146 form the lattice shape 140L on the exterior 130e of the balloon 130 of. In connecting to each other to form the lattice shape 140L, the plurality of branching drug tubes 146 may extend in any direction on the exterior 130e of the balloon 130. In FIG. 9B, the plurality of branching drug tubes 146 forming the lattice shape 140L connect with each other at approximately regular lengths. However, in other embodiments, the plurality of branching drug tubes 146 forming the lattice shape 140L may connect with each other at varying lengths. In FIG. 9B, the plurality of branching drug tubes 146 forming the lattice shape 140L have approximately similar lengths. However, in other embodiments, the plurality of branching drug tubes 146 forming the lattice shape 140L may have varying lengths. In FIG. 9B, the plurality of branching drug tubes 146 forming the lattice shape 140L have approximately straight shapes. However, in other embodiments, the plurality of branching drug tubes 146 forming the lattice shape 140L may be curved or bended.

### Second Embodiment

FIG. 10A-11B depict a drug eluting apparatus 200 (e.g., treatment apparatus, etc.) for delivering a drug (e.g., sirolimus, etc.) into a vessel according to a second embodiment. The drug eluting apparatus 200 includes a handle 206 including a drug port 204 configured to receive a drug. The drug eluting apparatus 200 also includes a carrier tube 260 connected to the handle 206, the carrier tube 260 having a lumen 262. The drug eluting apparatus 200 also includes a drug tube 240 configured to move through the lumen 262 of the carrier tube 260. The drug tube 240 includes a proximal portion 240p configured to receive a drug from the drug port 204 and a distal portion 240d including a plurality of holes configured to allow the drug to exit the drug tube. When the distal portion 240d of the drug tube 240 is located in the carrier tube, the distal portion 240d of the drug tube 240 is held by the carrier tube 260 in a deformed radially collapsed state. When the distal portion 240d of the drug tube 240 is outside the carrier tube 260, the distal portion 240d of the drug tube 240 takes on a relaxed radially expanded state 244a. In some embodiments, the handle 206 comprises an actuator 208, the actuator 208 configured to move the drug tube 240 relative to the carrier tube 260 or to move the carrier tube 260 relative to the drug tube 240. In some embodiments, the drug eluting apparatus 200 may also include a radiopaque marker 270.

The drug eluting apparatus 200 includes a handle 206 (e.g., grip, handgrip, etc.). The handle 206 is configured in the same way as the handle 106. The handle 206 includes a drug port 204 (e.g., hole, opening, aperture, etc.) configured to receive a drug. The drug port 204 is configured in the same way as the drug port 104. In other embodiments, the drug port 104 may be located on a proximal portion 240p of the drug tube 240.

In some embodiments, the handle 206 includes an actuator 208 (e.g., tab, button, wheel, knob, etc.). The actuator 208 is configured in the same way as the actuator 108. In some embodiments, the actuator 208 is configured to move the drug tube 240 relative to the carrier tube 260. The drug tube 240 may be rotated in a clockwise or a counterclockwise direction about the longitudinal axis A_{L} and/or may be moved in a longitudinal direction along the longitudinal axis A_{L}. In other embodiments, the actuator 208 is configured to move the carrier tube 260 relative to the drug tube 240. The carrier tube 260 may be rotated in a clockwise or a counterclockwise direction about the longitudinal axis A_{L} and/or may be moved in a longitudinal direction along the longitudinal axis A_{L}.

The drug eluting apparatus also includes a carrier tube 260 (e.g., a sheath, guiding sheath, cover, etc.) configured to allow the drug tube 140 to move therethrough. The carrier tube 260 is configured in the same way as the carrier tube 160. The carrier tube 260 has a lumen 262 (e.g., cavity, space, etc.). The carrier tube 260 may be a long carrier tube 180 (discussed in further detail herein), as in FIG. 10A. The carrier tube 260 is connected to or connectable to the handle 206. When the carrier tube 260 is connected to the handle 206, the carrier tube 260 is connected to the handle 206 by a Luer lock connector, a lock connector, a fitting connector, or by other suitable connecting arrangements. In other embodiments, the carrier tube 260 may be a short carrier tube 182 (discussed in further detail herein). In some embodiments, the carrier tube 260 is configured to be attached to a guiding catheter 184 (discussed in further detail herein).

The drug eluting apparatus also includes a drug tube 240 (e.g., pipe, cylinder, conduit, etc.). The drug tube 240 is configured in the same way as the drug tube 140. The drug tube 240 is configured to move through the lumen 262 of the carrier tube 260. At least the distal portion 240d of the drug tube 240 may be made of an elastically deformable material. In some embodiments, the drug tube 240 may be made of nitinol or stainless steel.

The drug tube 240 comprises a proximal portion 240p and a distal portion 240d. The proximal portion 240p of the drug tube 240 is configured to receive the drug from the drug port 204.

The distal portion 240d of the drug tube 240 includes a plurality of holes 242 (e.g., apertures, openings, slits, etc.). The plurality of holes 242 are configured in the same way as the plurality of holes 142. In some embodiments, the plurality of holes 242 are positioned on the drug tube 240 in a radially outward direction RO relative to a longitudinal axis A_{L} of the carrier tube 260, as shown in FIG. 10A.

When the distal portion 240d of the drug tube 240 is located in the carrier tube 260, the distal portion 240d of the drug tube 240 is held by the carrier tube 260 in the deformed radially collapsed state, and when the distal portion 240d of the drug tube 240 is outside the carrier tube 260, the distal portion 240d of the drug tube 240 takes on the relaxed radially expanded state 244a as the carrier tube 260 no longer applies an external force to the distal portion 240d of the drug tube 240. The deformed state is the state in which an external force is applied to the distal portion 240d of the drug tube 240. The relaxed state is the state in which no external force is applied to the distal portion 240d of the drug tube 240.

The drug eluting apparatus 200 may be used with or without a guidewire. The guidewire may be configured in the same way as the guidewire 126.

In the embodiment of FIG. 10A and 10B, the distal portion 240d of the drug tube 240 extends in a helical shape 240h when the distal portion 240d of the drug tube 240 is in the relaxed radially expanded state 244a. In a helical shape 240h, the distal portion 240d of the drug tube 240 has a shape that consists of a series of rotations along the distal portion 240d of the drug tube 240 from a proximal end 241p of the distal portion 240d of the drug tube 240 to the distal end 241d of the distal portion 240d of the drug tube 240.

The rotations of the helical shape 240h of the distal portion 240d of the drug tube 240 may be circular, elliptical, or other rounded arrangements. In FIG. 10A and 10B, the rotations of the helical shape 240h of the distal portion 240d of the drug tube 240 have the same spacing in between each rotation. However, in other embodiments, the rotations of the helical shape 240h of the distal portion 240d of the drug tube 240 may have varying spacing in between each rotation.

In the embodiment of FIG. 11A and 11B, when the distal portion 240d of the drug tube 240 is in the relaxed radially expanded state 244a, the distal portion 240d of the drug tube 240 includes a plurality of drug tube portions 246. The plurality of drug tube portions 246 extend from a proximal end 241p of the distal portion 240d of the drug tube 240 to a distal end 241d of the distal portion 240d of the drug tube 240 and are curved radially outward in a direction RO relative to a longitudinal axis A_{L} of the carrier tube 260. In FIG. 11A and 11B, the plurality of drug tube portions 246 are each shown as having an approximately similar width. However, in other embodiments, the plurality of drug tube portions 246 may have varying widths. In FIG. 11A, the plurality of drug tube portions 246 are shown as having six drug tube portions. In FIG. 11B, the plurality of drug tube portions 246 are shown as having nine drug tube portions. In other embodiments, the plurality of drug tube portions 246 may have one, two, three, four, five, six, seven, eight, nine, or more drug tube portions.

In some embodiments, the drug eluting apparatus may further include a radiopaque marker 270 (e.g., hoop, band, disk, etc.). In FIG. 10B and 11B, the radiopaque marker 270 is connected to the drug tube 240. The radiopaque marker 270 may be connected the drug tube 240 by blading, by swaging, by press-fit, by welding, or by other suitable connecting arrangements. The radiopaque marker 270 has a maximum cross-sectional width that is approximately in a range of 1-15 mm, inclusive, and for example, 3-8 mm.

The radiopaque marker 270 may be made of gold, platinum, stainless steel, tungsten, tantalum, or other suitable materials. The radiopaque marker 270 may be made of a radiopaque material. When the radiopaque marker 270 is made of a radiopaque material, the radiopaque marker 270 may be imaged using an imaging modality such as X-ray, fluoroscopy, or other suitable imaging modality. The radiopaque material of the radiopaque marker 270 facilitates the detection and tracking via imaging of the drug tube 240 when the drug tube 240 is inside a vessel. Such detection and tracking of the radiopaque marker 270 may allow for improved operation and positioning of the drug eluting apparatus 200 within the vessel and for improved targeting of the location of a portion of a vessel, as a user may receive information of where the drug tube 240 is located through knowledge of the location of the radiopaque marker 270 via imaging.

In FIG. 10B and 11B, the drug eluting apparatus 200 has two radiopaque markers 270, with one radiopaque marker 270 connected to the distal portion 240d of the drug tube 240, and another radiopaque marker 270 connected to the proximal portion of the drug tube 240. In other embodiments, the drug eluting apparatus 200 may have one radiopaque marker 270. In some embodiments, the drug eluting apparatus 200 that has one radiopaque marker 270 may have the radiopaque marker 270 connected to the distal portion 240d of the drug tube 240. In other embodiments, the drug eluting apparatus 200 that has one radiopaque marker 270 may have the radiopaque marker 270 connected to the proximal portion 240p of the drug tube 240.

### General Operation of Drug Eluting Apparatuses, and Long and Short Carrier Tubes

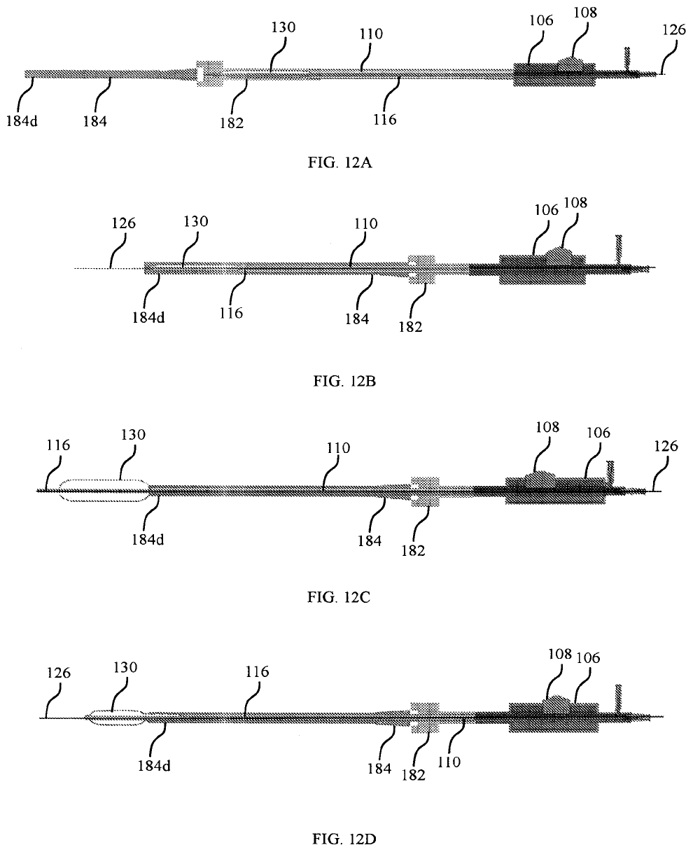

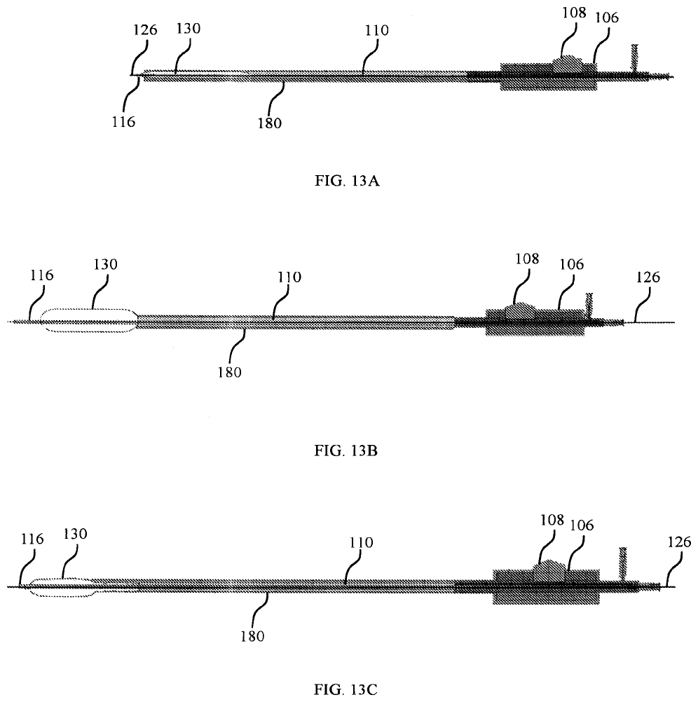

FIG. 12A-14D depict drug eluting apparatuses in different configurations of operation of the drug eluting apparatuses. The drug eluting apparatuses may be used with a needle, a trocar, a guiding catheter, a short carrier tube, a long carrier tube, an inserter, a valve, and/or a flushing device.

The drug eluting apparatuses 100 and 200 may be used with a needle for insertion into a subject. The needle may be used to create a hole in the subject that provides access to a vessel. The vessel that the needle provides access to may be the vessel that is the target of drug elution or may be a vessel through which a drug eluting apparatus may be pushed to reach another vessel that is the target of drug elution. In other embodiments, a trocar may be used to create the hole in the subject that provides access to the vessel.

After the needle has provided access to a vessel, a guidewire, which may be the guidewire 126, may be pushed through the needle to enter the vessel and positioned at or near the target of drug elution. Imaging may be used to facilitate positioning the guidewire in the subject. The needle is then be withdrawn from the subject, and the guidewire is maintained in the subject. The guidewire is used to insert a portion of the drug eluting apparatuses 100 or 200 into the subject. The guidewire may then be withdrawn from the vessel, and the components passed over the guidewire are maintained in the vessel. In other embodiments, the guidewire may remain in the vessel, and after the drug eluting apparatus 100 or 200 is used and withdrawn, the guidewire is withdrawn.

The drug eluting apparatuses 100 and 200 may be used with a guiding catheter 184 (e.g., catheter, shaft, tube, pipe, cylinder, conduit, etc.). The guiding catheter 184 is passed over a guidewire and inserted into the vessel. The guiding catheter 184 may be positioned at or near the target of drug elution. In some embodiments, the shaft 110, the balloon 130, the drug tube 140, and/or the carrier tube 160 may be passed through the guiding catheter 184. In other embodiments, the drug tube 240 and/or the carrier tube 260 may be passed through the guiding catheter.

Use of a guiding catheter 184 allows the balloon 130 and the drug tube 140 or the drug tube 240 to advance through the vessel without encountering calcifications, plaques, or other materials in the vessel that could interrupt the advancement of the balloon 130 and the drug tube 140 or the drug tube 240 if otherwise encountered in the absence of a guiding catheter 184.

FIG. 12A-14D depict drug eluting apparatuses with long carrier tubes 180 (e.g., a sheath, guiding sheath, cover, etc.) or short carrier tubes 182 (e.g., a sheath, guiding sheath, cover, etc.). In some embodiments, a long carrier tube 180 may be a carrier tube 160 having a length that is equal to or greater than a length of the shaft 110. In other embodiments, a long carrier tube 180 may be a carrier tube 260 having a length that is equal to or greater than an axial length 250 of the drug tube 240. The axial length 250 of the drug tube 240 is measured along the longitudinal axis A_{L} as in FIG. 10A. In some embodiments, a short carrier tube 182 may be a carrier tube 160 having a length that is approximately equal to an axial length 151 of the distal portion 140d of the drug tube 140, in a range of approximately 100-120% of the axial length of the distal portion 140d of the drug tube 140, and preferably 110%. The axial length 151 of the distal portion 140d of the drug tube 140 is measured along the longitudinal axis A_{L} as in FIG. 2A. In other embodiments, a short carrier tube 182 may be a carrier tube 260 having a length that is approximately equal to an axial length 251 of the distal portion 240d of the drug tube 240, in a range of approximately 100-120% of the axial length of the distal portion 240d of the drug tube 240, and preferably 110%. The axial length 251 of the distal portion 240d of the drug tube 240 is measured along the longitudinal axis A_{L} as in FIG. 10A. Long carrier tubes 180 and short carrier tubes 182 may be used with the guiding catheter 184.

FIG. 12A-12D depict a drug eluting apparatus with a short carrier tube 182 and a guiding catheter 184. The short carrier tube 182 may connect with the guiding catheter 184 such that a shaft 110 or the drug tube 240 may pass first through the short carrier tube 182 and second through the guiding catheter 184. The short carrier tube 182 may connect with the guiding catheter 184 by snap fit, by a Luer lock connector, or by other suitable connecting arrangements. The short carrier tube 182 may connect to a handle, which may be the handle 106 or the handle 206, by a Luer lock connector, a lock connector, a fitting connector, or by other suitable connecting arrangements. In the configuration of FIG. 12A, the short carrier tube 182 is not connected to the handle 106. In the configurations of FIG. 12B-12D, the short carrier tube 182 is connected to the handle 106.

Use of a short carrier tube 182 and a guiding catheter 184 allows for protection of the balloon 130 during insertion of the balloon 130 and allows the balloon 130 to be primed and then directly inserted into the guiding catheter 184, which eliminates the introduction of air in the guiding catheter 184. Use of a short carrier tube 182 facilitates transfer of the balloon 130 to the guiding catheter 184.

In FIG. 12A-12D, the short carrier tube 182 is shown with the drug eluting apparatus 100. In FIG. 12A, the balloon 130 and the drug tube 140 are positioned in the short carrier tube 182. In FIG. 12B, the balloon 130 and the drug tube 140 have been pushed through the short carrier tube 182 and into the guiding catheter 184. In some embodiments, in which the drug eluting apparatus 100 has a balloon 130 and the relaxed state of the distal portion 140d of the drug tube 140 is the radially expanded state 144a, as the balloon 130 and distal portion 140d of the drug tube 140 are being pushed outside of the guiding catheter 184, the distal portion 140d of the drug tube 140 is partially expanded and the balloon 130 is partially inflated, similar to the shape of the balloon 130 in FIG. 12D. In FIG. 12C, the balloon 130 and the drug tube 140 have been pushed forward such that the balloon 130 and the distal portion 140d of the drug tube 140 are fully outside of the guiding catheter 184. In FIG. 12C, the balloon 130 is inflated, and the distal portion 140d of the drug tube 140 is in the deformed radially expanded state 144a or the relaxed radially expanded state 144a. In FIG. 12D, the balloon 130 and the drug tube 140 are being pulled back such that the balloon 130 and the distal portion 140d of the drug tube 140 are partially inside of the guiding catheter 184. After the drug has been delivered, the balloon 130 and the distal portion 140d of the drug tube 140 may be pulled back such that the balloon 130 and the distal portion 140d of the drug tube are inside of the guiding catheter 184, as in FIG. 12B, or in the short carrier tube 182, as in FIG. 12A.

In other embodiments, the short carrier tube 182 of FIG. 12A-12D, may be used with the drug eluting apparatus 200. FIG. 12A-12D depict an over-the-wire type device. In other embodiments, the short carrier tube 182 of FIG. 12A-12D may be used with a rapid-exchange type device. When the short carrier tube 182 is used with a rapid-exchange type device and the guiding catheter 184, the guidewire 126 is positioned alongside the shaft 110 and inside the guiding catheter 184.

In FIG. 13A-13C, the long carrier tube 180 is shown with the drug eluting apparatus 100. The long carrier tube 180 connects to a handle, which may be the handle 106 or the handle 206, and is connected by a Luer lock connector, a lock connector, a fitting connector, or by other suitable connecting arrangements. In FIG. 13A, the balloon 130 and the drug tube 140 are positioned in the long carrier tube 180. In some embodiments, in which the drug eluting apparatus 100 has a balloon 130 and the relaxed state of the distal portion 140d of the drug tube 140 is the radially expanded state 144a, as the balloon 130 and distal portion 140d of the drug tube 140 are being pushed outside of the long carrier tube 180, the distal portion 140d of the drug tube 140 is partially expanded and the balloon 130 is partially inflated, similar to the shape of the balloon 130 in FIG. 13C. In FIG. 13B, the balloon 130 and the drug tube 140 have been pushed forward such that the balloon 130 and the distal portion 140d of the drug tube 140 are fully outside of the long carrier tube 180. In FIG. 13C, the balloon 130 is inflated, and the distal portion 140d of the drug tube 140 is in the deformed radially expanded state 144a or the relaxed radially expanded state 144a. In FIG. 13C, the balloon 130 and the drug tube 140 are being pulled back such that the balloon 130 and the distal portion of the drug tube 140 are partially inside of the long carrier tube 180. After the drug has been delivered, the balloon 130 and the distal portion 140d of the drug tube 140 may be pulled back such that the balloon 130 and the distal portion 140d of the drug tube are inside of the long carrier tube 180, as in FIG. 13A.

In other embodiments, the long carrier tube 180 of FIG. 13A-13C may be used with the drug eluting apparatus 200. In some embodiments, the long carrier tube 180 of FIG. 13A-13C may be used with the guiding catheter 184 of FIG. 12A-12D in place of the short carrier tube 182. FIG. 13A-13C depict an over-the-wire type device. In other embodiments, the long carrier tube 180 of FIG. 13A-13C may be used with a rapid-exchange type device. When the long carrier tube 180 is used with a rapid-exchange type device and the guiding catheter 184, the guidewire 126 is positioned alongside the shaft 110 and inside the guiding catheter 184.

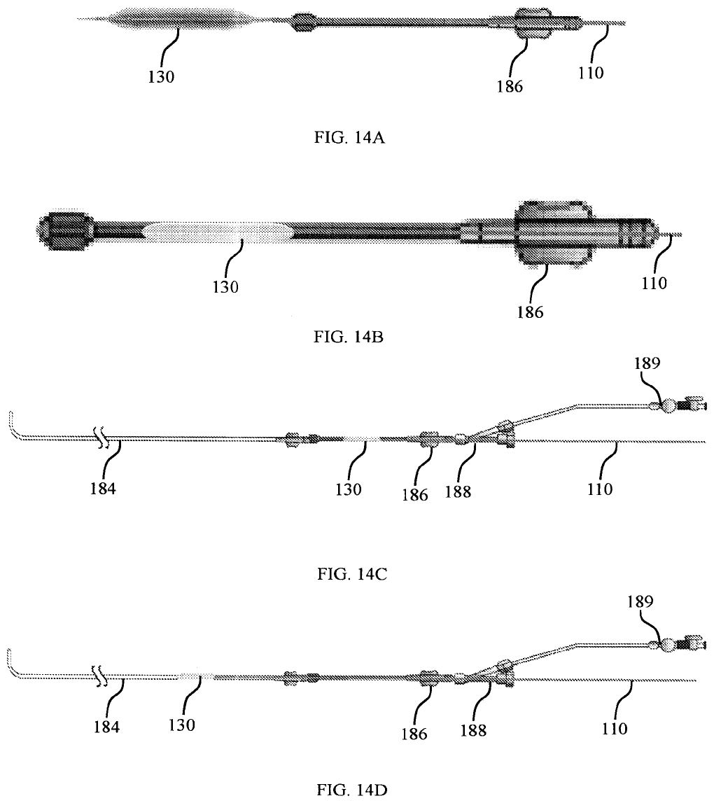

FIG. 14A-14D depict a drug eluting apparatus with the guiding catheter 184, an inserter 186 (e.g., shaft, tube, pipe, cylinder, conduit, etc.), a valve 188 (e.g., a connection, a gate, a tap, etc.), and a flushing device 189. The guiding catheter 184 may be connected to the inserter 186 by a Luer lock connector or by other suitable connecting arrangements.

The inserter 186 may be connected to the valve 188 by a Luer lock connector or by other suitable connecting arrangements. The inserter 186 may be passed over the guidewire. The use of an inserter 186 to provide the balloon 130 to the guiding catheter 184 may facilitate the pushing of the balloon 130 in the guiding catheter 184 as without an inserter 186 the balloon 130 may buckle when pushed or pushing the balloon 130 may introduce air into the guiding catheter 184.

In FIG. 14C and 14D, the valve 188 is a three-way valve configured to connect to the inserter 186, to connect to a flushing device 189, and to receive a portion of the drug eluting apparatus 100 or the drug eluting apparatus 200. In other embodiments, the valve 188 may be a two-way valve configured to connect to the inserter 186 and to receive a portion of the drug eluting apparatus 100 or the drug eluting apparatus 200. The valve 188 may be passed over the guidewire.

The flushing device 189 may be used to flush the inserter 186 and/or the guiding catheter 184 so that the inserter 186 is de-aired, which allows for an easier and safer transition of the balloon 130 into the guiding catheter 184 without air.

In FIG. 14A-14D, the guiding catheter 184, an inserter 186, and a valve 188 are shown with a portion of the drug eluting apparatus 100. In FIG. 14A, the shaft 110 is pulled through the inserter 186. In FIG. 14B, the shaft 110 is pulled through the inserter 186 such that the balloon 130 is in the inserter 186. In FIG. 14C the shaft 110 is pulled such that the shaft 110 passes through the valve 188. In FIG. 14C, the inserter 186 is shown as connected to the guiding catheter 184 and also connected to the valve 188. Before or after use of the inserter 186, guiding catheter 184, and valve 188, the inserter 186 may be unconnected to the guiding catheter 184 and also unconnected to the valve 188. The inserter 186 may be connected to the guiding catheter 184 by a Luer lock connector, or by other suitable connecting arrangements. The inserter 186 may be connected to the valve 188 by a Luer lock connector, or by other suitable connecting arrangements. In FIG. 14D, the balloon 130 is pushed such that the balloon 130 and the shaft 110 enter the guiding catheter 184.

In other embodiments, the guiding catheter 184, the inserter 186, the valve 188, and/or the flushing device 189 of FIG. 14A-14D may be used with the drug eluting apparatus 200. In other embodiments, the inserter 186, the valve 188, and/or the flushing device 189 of FIG. 14A-14D may be used with the long carrier tube 180 or the short carrier tube 182. In some embodiments, the inserter 186, the valve 188, and/or the flushing device 189 may be used with a rapid-exchange type device. In other embodiments, the inserter 186, the valve 188, and/or the flushing device 189 may be used with an over-the-wire type device.

### Methods of Delivering Drugs Using Drug Eluting Apparatuses

An example first method for delivering a drug into a vessel includes providing a drug eluting apparatus 100. The first method also includes inserting the distal portion 110d of the shaft 110 into a vessel. The first method also includes inflating the balloon 130 by providing the inflation fluid to the balloon 130 via the balloon inflation tube 112, which causes the distal portion 140d of the drug tube 140 to take a deformed radially expanded state 144a. The first method also includes delivering the drug into the vessel via the drug tube 140 and the plurality of holes 142 of the drug tube 140.

The first method includes providing a drug eluting apparatus 100. The drug eluting apparatus 100 includes a handle 106 including a balloon inflation port 102 configured to receive an inflation fluid and a drug port 104 configured to receive a drug. The drug eluting apparatus 100 also includes a shaft 110 connected to the handle 106. The shaft 110 includes a balloon inflation lumen 113 configured to receive the inflation fluid from the balloon inflation port 102, a drug lumen 114 configured to receive the drug from the drug port 104, and a guidewire lumen 117 configured to receive a guidewire 126 and allow the guidewire 126 to extend out of a distal portion 110d of the shaft 110. The drug eluting apparatus 100 also includes a balloon 130 attached to the distal portion 110d of the shaft 110 and configured to receive the inflation fluid from the balloon inflation lumen 113. The drug eluting apparatus 100 also includes a drug tube 140 defining a distal portion 114d of the drug lumen 114, the drug tube 140 having a distal portion 140d connected to an exterior 130e of the balloon 130 and including a plurality of holes 142 configured to allow the drug to exit the drug tube 140. In some embodiments, the drug eluting apparatus 100 may further include a guidewire tube 116 in which the guidewire lumen 117 is formed. In some embodiments, the drug eluting apparatus 100 may further include a balloon inflation tube 112 in which the balloon inflation lumen 113 is formed. While the first method is described with respect to the drug eluting apparatus 100, it should be appreciated that the operations of the first method are equally applicable to any other apparatus that includes components analogous to those described therein.

The first method includes inserting the distal portion 110d of the shaft 110 into a vessel, such that the distal portion 110d of the shaft 110 is at a location in the vessel that is a target of drug elution. Inserting the distal portion 110d of the shaft 110 into a vessel may include using a needle, a trocar, a guiding catheter 184 as in FIG. 12A-12D, an inserter 186 as in FIG. 14A-14D, a valve 188 as in FIG. 14A-14D, and/or a flushing device 189 as in FIG. 14A-14D.

The first method includes inflating the balloon 130 by providing the inflation fluid to the balloon 130 via the balloon inflation tube 112, which causes the distal portion 140d of the drug tube 140 to take the deformed radially expanded state 144a, as in FIG. 1, 2A, 2B, 8, or 9A. Pressure to the inflated balloon 130 is maintained by the drug entering the balloon 130, which maintains the distal portion 140d of the drug tube 140 in its deformed radially expanded state 144a.

The first method includes delivering the drug into the vessel via the drug tube 140 and the plurality of holes 142 of the drug tube 140. The drug is provided via the drug port 104 to the drug tube 140.

An example second method for delivering a drug into a vessel includes providing a drug eluting apparatus 100 using the long carrier tube 180 as the carrier tube 160. The second method also includes inserting the distal portion 110d of the shaft 110 into a vessel. The second method also includes pushing the shaft 110 or pulling the carrier tube 160 so that the distal portion 140d of the drug tube 140 and the balloon 130 are outside of the carrier tube 160, which causes the distal portion 140d of the drug tube 140 to take a relaxed radially expanded state 144a. The second method also includes inflating the balloon 130 by providing the inflation fluid to the balloon 130 via the balloon inflation tube 112. The second method also includes delivering the drug into the vessel via the drug tube 140 and the plurality of holes 142 of the drug tube 140.

The second method includes providing a drug eluting apparatus 100. The drug eluting apparatus includes a handle 106 including a balloon inflation port 102 configured to receive an inflation fluid and a drug port 104 configured to receive a drug. The drug eluting apparatus 100 also includes a shaft 110 connected to the handle 106. The shaft 110 includes a balloon inflation lumen 113 configured to receive the inflation fluid from the balloon inflation port 102, a drug lumen 114 configured to receive the drug from the drug port 104, and a guidewire lumen 117 configured to receive a guidewire 126 and allow the guidewire 126 to extend out of a distal portion 110d of the shaft 110. The drug eluting apparatus 100 also includes a balloon 130 attached to the distal portion 110d of the shaft 110 and configured to receive the inflation fluid from the balloon inflation lumen 113. The drug eluting apparatus 100 also includes a carrier tube 160 connected to the handle 106, the carrier tube configured to allow the shaft 110 to move therethrough. The carrier tube 160 has a length that is equal to or greater than a length of the shaft 110. The drug eluting apparatus 100 also includes a drug tube defining a distal portion of the drug lumen, the drug tube having a distal portion 140d connected to an exterior 130e of the balloon 130 and including a plurality of holes 142 configured to allow the drug to exit the drug tube 140. In some embodiments, the handle 106 comprises an actuator 108, the actuator 108 configured to move the shaft 110 relative to the carrier tube 160 or to move the carrier tube 160 relative to the shaft 110. In some embodiments, the drug eluting apparatus 100 may further include a guidewire tube 116 in which the guidewire lumen 117 is formed. In some embodiments, the drug eluting 100 may further include a balloon inflation tube 112 in which the balloon inflation lumen 113 is formed. While the second method is described with respect to the drug eluting apparatus 100, it should be appreciated that the operations of the second method are equally applicable to any other apparatus that includes components analogous to those described therein.

The second method includes inserting the distal portion 110d of the shaft 110 into a vessel, such that the distal portion 110d of the shaft 110 is at a location in the vessel that is a target of drug elution. Inserting the distal portion 110d of the shaft 110 into a vessel may include using a needle, a trocar, a guiding catheter 184 as in FIG. 12A-12D, an inserter 186 as in FIG. 14A-14D, a valve 188 as in FIG. 14A-14D, and/or a flushing device 189 as in FIG. 14A-14D.

The second method includes pushing the shaft 110 or pulling the carrier tube 160 so that the distal portion 140d of the drug tube 140 and the balloon 130 are outside of the carrier tube 160, which causes the distal portion 140d of the drug tube 140 to take a relaxed radially expanded state 144a, as in FIG. 1, FIG. 2A, FIG. 2B, FIG. 8, or FIG. 9A. In some embodiments, the actuator 108 of the handle 106 may be used to push the shaft 110 or to pull the carrier tube 160. The carrier tube 160 holds the distal portion 140d of the drug tube 140 in a deformed radially compressed state, as in FIG. 12A or FIG. 13A, and removing the carrier tube 160 surrounding the distal portion 140d of the drug tube 140 allows the distal portion 140d of the drug tube 140 to enter the relaxed radially expanded state 144a, as in FIG. 12D or FIG. 13C.

The second method includes inflating the balloon 130 by providing the inflation fluid to the balloon 130 via the balloon inflation tube 112. The balloon 130 may be inflated before, simultaneously as, or after the distal portion 140d of the drug tube 140 takes on the relaxed radially expanded state 144a. The second method includes delivering the drug into the vessel via the drug tube 140 and the plurality of holes 142 of the drug tube 140. The drug is provided via the drug port 104 to the drug tube 140.

In some embodiments, the second method includes inserting a guiding catheter 184 into the vessel before inserting the distal portion 110d of the shaft 110 into the vessel. The carrier tube 160 is connected to the guiding catheter 184. In the step of inserting the distal portion 110d of the shaft 110 into the vessel, the distal portion 110d of the shaft 110 is inserted into the guiding catheter 184. In the step of pushing the shaft 110 or pulling the carrier tube 160, the shaft 110 is pushed so that the distal portion 140d of the drug tube 140 and the balloon 130 are outside of the guiding catheter 184.

An example third method for delivering a drug into a vessel includes providing a drug eluting apparatus 100 using a short carrier tube 182 as the carrier tube 160. The third method also includes inserting the distal portion 110d of the shaft 110, the carrier tube 160, and a guiding catheter 184 into a vessel. The third method also includes pushing the shaft 110 so that the distal portion 140d of the drug tube 140 and the balloon 130 are outside of the carrier tube 160 and the guiding catheter 184, which causes the distal portion 140d of the drug tube 140 to take a relaxed radially expanded state 144a. The third method also includes inflating the balloon 130 by providing the inflation fluid to the balloon 130 via the balloon inflation tube 112. The third method also includes delivering the drug into the vessel via the drug tube 140 and the plurality of holes 142 of the drug tube 140.

The third method includes providing a drug eluting apparatus 100. The drug eluting apparatus includes a handle 106 including a balloon inflation port 102 configured to receive an inflation fluid and a drug port 104 configured to receive a drug. The drug eluting apparatus 100 also includes a shaft 110 connected to the handle 106. The shaft 110 includes a balloon inflation lumen 113 configured to receive the inflation fluid from the balloon inflation port 102, a drug lumen 114 configured to receive the drug from the drug port 104, and a guidewire lumen 117 configured to receive a guidewire 126 and allow the guidewire 126 to extend out of a distal portion 110d of the shaft 110. The drug eluting apparatus 100 also includes a balloon 130 attached to the distal portion 110d of the shaft 110 and configured to receive the inflation fluid from the balloon inflation lumen 113. The drug eluting apparatus 100 also includes a carrier tube 160 connected to or connectable to the handle 106, the carrier tube 160 configured to allow the shaft 110 to move therethrough. The carrier tube 160 is configured to be attached to a guiding catheter 184. The drug eluting apparatus 100 also includes a drug tube defining a distal portion of the drug lumen, the drug tube having a distal portion 140d connected to an exterior 130e of the balloon 130 and including a plurality of holes 142 configured to allow the drug to exit the drug tube 140. The carrier tube 160 has a length that is approximately equal to an axial length of the distal portion 140d of the drug tube 140. In some embodiments, the handle 106 comprises an actuator 108, the actuator 108 configured to move the shaft 110 relative to the carrier tube 160 or to move the carrier tube 160 relative to the shaft 110. In some embodiments, the drug eluting 100 may further include a guidewire tube 116 in which the guidewire lumen 117 is formed. In some embodiments, the drug eluting 100 may further include a balloon inflation tube 112 in which the balloon inflation lumen 113 is formed. While the third method is described with respect to the drug eluting apparatus 100, it should be appreciated that the operations of the third method are equally applicable to any other apparatus that includes components analogous to those described therein.

The third method includes inserting the distal portion 110d of the shaft 110, the carrier tube 160, and the guiding catheter 184 into a vessel, such that the distal portion 110d of the shaft 110 is at a location in the vessel that is a target of drug elution. Inserting the distal portion 110d of the shaft 110 into a vessel may include using a needle, a trocar, an inserter 186 as in FIG. 14A-14D, a valve 188 as in FIG. 14A-14D, and/or a flushing device 189 as in FIG. 14A-14D.

The third method includes pushing the shaft 110 so that the distal portion 140d of the drug tube 140 and the balloon 130 are outside of the carrier tube 160 and the guiding catheter 184, which causes the distal portion 140d of the drug tube 140 to take a relaxed radially expanded state 144a, as in FIG. 1, FIG. 2A, FIG. 2B, FIG. 8, or FIG. 9A. In some embodiments, the actuator 108 of the handle 106 may be used to push the shaft 110. The carrier tube 160 or the guiding catheter 184 holds the distal portion 140d of the drug tube 140 in a deformed radially compressed state, as in FIG. 12A or FIG. 13A, and removing the carrier tube 160 or the guiding catheter 184 surrounding the distal portion 140d of the drug tube 140 allows the distal portion 140d of the drug tube 140 to enter the relaxed radially expanded state 144a, as in FIG. 12D or FIG. 13C.

The third method includes inflating the balloon 130 by providing the inflation fluid to the balloon 130 via the balloon inflation tube 112. The balloon 130 may be inflated before, simultaneously as, or after the distal portion 140d of the drug tube 140 takes on the relaxed radially expanded state 144a.

The third method includes delivering the drug into the vessel via the drug tube 140 and the plurality of holes 142 of the drug tube 140. The drug is provided via the drug port 104 to the drug tube 140.

An example fourth method for delivering a drug into a vessel includes providing a drug eluting apparatus 200 using a long carrier tube 180 as the carrier tube 260. The fourth method also includes inserting the distal portion 260d of the carrier tube 260 into a vessel. The fourth method also includes pushing the drug tube 240 or pulling the carrier tube 260 so that the distal portion 240d of the drug tube 240 is outside of the carrier tube 260, which causes the distal portion 240d of the drug tube 240 to take a relaxed radially expanded state 244a, the drug tube 240 receiving the drug from the drug port 204. The fourth method also includes delivering the drug into the vessel via the drug tube 240 and the plurality of holes 242 of the drug tube 240.

The fourth method includes providing a drug eluting apparatus 200. The drug eluting apparatus includes a handle 206 including a drug port 204 configured to receive a drug. The drug eluting apparatus 200 also includes a carrier tube 260 connected to the handle 206, the carrier tube 260 having a lumen 262. The drug eluting apparatus 200 also includes a drug tube 240 configured to move through the lumen 262 of the carrier tube 260. The drug tube 240 includes a proximal portion 240p configured to receive a drug from the drug port 204 and a distal portion 240d including a plurality of holes configured to allow the drug to exit the drug tube. When the distal portion 240d of the drug tube 240 is located in the carrier tube, the distal portion 240d of the drug tube 240 is held by the carrier tube 260 in a deformed radially collapsed state. When the distal portion 240d of the drug tube 240 is outside the carrier tube 260, the distal portion 240d of the drug tube 240 takes on a relaxed radially expanded state 244a. The carrier tube 260 has a length that is equal to or greater than an axial length of the drug tube 240. In some embodiments, the handle 206 comprises an actuator 208, the actuator 208 configured to move the drug tube 240 relative to the carrier tube 260 or to move the carrier tube 260 relative to the drug tube 240. In some embodiments, the drug eluting apparatus 200 may further include a radiopaque marker 270. While the fourth method is described with respect to the drug eluting apparatus 200, it should be appreciated that the operations of the fourth method are equally applicable to any other apparatus that includes components analogous to those described therein.

The fourth method includes inserting the distal portion 260d of the carrier tube 260 into a vessel, such that the distal portion 260d of the carrier tube 260 is at a location in the vessel that is a target of drug elution. Inserting the distal portion 260d of the carrier tube 260 into a vessel may include using a needle, a trocar, a guiding catheter 184 as in FIG. 12A-12D, an inserter 186 as in FIG. 14A-14D, a valve 188 as in FIG. 14A-14D, and/or a flushing device 189 as in FIG. 14A-14D.

The fourth method includes pushing the drug tube 240 or pulling the carrier tube 260 so that the distal portion 240d of the drug tube 240 is outside of the carrier tube 260, which causes the distal portion 240d of the drug tube 240 to take a relaxed radially expanded state 244a, as in FIG. 10A, FIG. 10B, FIG. 11A, or FIG. 11B. In some embodiments, the actuator 208 of the handle 206 may be used to push the shaft 210 or to pull the carrier tube 260. When the carrier tube 260 surrounds the distal portion 240d of the drug tube 240, the carrier tube 260 holds the distal portion 240d of the drug tube 240 in a deformed radially compressed state, and removing the carrier tube 260 surrounding the distal portion 240d of the drug tube 240 allows the distal portion 240d of the drug tube 240 to enter the relaxed radially expanded state 244a.

The fourth method includes delivering the drug into the vessel via the drug tube 240 and the plurality of holes 242. The drug is provided via the drug port 204 to the drug tube 240.

In some embodiments, the second method includes inserting a guiding catheter 184 into the vessel before inserting the distal portion 110d of the shaft 110 into the vessel. The carrier tube 160 is connected to the guiding catheter 184. In the step of inserting the distal portion 110d of the shaft 110 into the vessel, the distal portion 110d of the shaft 110 is inserted into the guiding catheter 184. In the step of pushing the shaft 110 or pulling the carrier tube 160, the shaft 110 is pushed so that the distal portion 140d of the drug tube 140 and the balloon 130 are outside of the guiding catheter 184.

An example fifth method for delivering a drug into a vessel includes providing a drug eluting apparatus 200 using a short carrier tube 182 as the carrier tube 260. The fifth method also includes inserting the distal portion 260d of the carrier tube 260 and a guiding catheter 184 into a vessel. The fifth method also includes pushing the drug tube 240 so that the distal portion 240d of the drug tube 240 is outside of the carrier tube 260 and the guiding catheter 184, which causes the distal portion 240d of the drug tube 240 to take a relaxed radially expanded state 244a, the drug tube 240 receiving the drug from the drug port 204. The fifth method also includes delivering the drug into the vessel via the drug tube 240 and the plurality of holes 242 of the drug tube 240.

The fifth method includes providing a drug eluting apparatus 200. The drug eluting apparatus includes a handle 206 including a drug port 204 configured to receive a drug. The drug eluting apparatus 200 also includes a carrier tube 260, the carrier tube 260 connected to or connectable to the handle 206. The carrier tube 260 has a lumen 262. The carrier tube 260 is configured to be attached to a guiding catheter 184. The drug eluting apparatus 200 also includes a drug tube 240 configured to move through the lumen 262 of the carrier tube 260. The drug tube 240 includes a proximal portion 240p configured to receive a drug from the drug port 204 and a distal portion 240d including a plurality of holes configured to allow the drug to exit the drug tube. When the distal portion 240d of the drug tube 240 is located in the carrier tube, the distal portion 240d of the drug tube 240 is held by the carrier tube 260 in a deformed radially collapsed state. When the distal portion 240d of the drug tube 240 is outside the carrier tube 260, the distal portion 240d of the drug tube 240 takes on a relaxed radially expanded state 244a. The carrier tube 260 has a length that is approximately equal to an axial length of the distal portion 240d of the drug tube 240. In some embodiments, the handle 206 comprises an actuator 208, the actuator 208 configured to move the drug tube 240 relative to the carrier tube 260 or to move the carrier tube 260 relative to the drug tube 240. In some embodiments, the drug eluting apparatus 200 may further include a radiopaque marker 270. While the fifth method is described with respect to the drug eluting apparatus 200, it should be appreciated that the operations of the fifth method are equally applicable to any other apparatus that includes components analogous to those described therein.

The fifth method includes inserting a distal portion 260d of the carrier tube 260 and the guiding catheter 184 into a vessel, such that a distal portion 184d of the guiding catheter 184 is at a location in the vessel that is a target of drug elution. Inserting the distal portion 260d of the carrier tube 260 and the guiding catheter 184 into a vessel may include using a needle, a trocar, an inserter 186 as in FIG. 14A-14D, a valve 188 as in FIG. 14A-14D, and/or a flushing device 189 as in FIG. 14A-14D.

The fifth method includes pushing the drug tube 240 so that the distal portion 240d of the drug tube 240 is outside of the carrier tube 260 and the guiding catheter 184, which causes the distal portion 240d of the drug tube 240 to take a relaxed radially expanded state 244a, as in FIG. 10A, FIG. 10B, FIG. 11A, or FIG. 11B. In some embodiments, the actuator 208 of the handle 206 may be used to push the shaft 210. When the carrier tube 260 or the guiding catheter 184 surrounds the distal portion 240d of the drug tube 240, the carrier tube 260 or the guiding catheter 184 holds the distal portion 240d of the drug tube 240 in a deformed state radially compressed state, and removing the carrier tube 260 or the guiding catheter 184 surrounding the distal portion 240d of the drug tube 240 allows the distal portion 240d of the drug tube 240 to enter the relaxed radially expanded state 244a.

The fifth method includes delivering the drug into the vessel via the drug tube 240 and the plurality of holes 242. The drug is provided via the drug port 204 to the drug tube 240.

### Construction of Example Embodiments

It should be noted that the term “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Additionally, it should be understood that features from one embodiment disclosed herein may be combined with features of other embodiments disclosed herein as one of ordinary skill in the art would understand. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, the use of ranges of values (e.g., W1 to W2, etc.) herein are inclusive of their maximum values and minimum values (e.g., W1 to W2 includes W1 and includes W2, etc.), unless otherwise indicated. Furthermore, a range of values (e.g., W1 to W2, etc.) does not necessarily require the inclusion of intermediate values within the range of values (e.g., W1 to W2 can include only W1 and W2, etc.), unless otherwise indicated.

1. A drug eluting apparatus comprising:
a handle comprising:
a balloon inflation port configured to receive an inflation fluid, and
a drug port configured to receive a drug;
a shaft connected to the handle, the shaft comprising:
a balloon inflation lumen configured to receive the inflation fluid from the balloon inflation port,
a drug lumen configured to receive the drug from the drug port, and
a guidewire lumen configured to receive a guidewire and allow the guidewire to extend out of a distal portion of the shaft;
a balloon attached to the distal portion of the shaft and configured to receive the inflation fluid from the balloon inflation lumen;
a drug tube defining a distal portion of the drug lumen, the drug tube having a distal portion connected to an exterior of the balloon and comprising a plurality of holes configured to allow the drug to exit the drug tube, wherein at least the distal portion of the drug tube is made of an elastically deformable material; and
a carrier tube connected to or connectable to the handle, the carrier tube being configured to allow the shaft to move therethrough; wherein:
when the balloon and the distal portion of the drug tube are located in the carrier tube, the distal portion of the drug tube is held by the carrier tube in a deformed radially collapsed state, and when the balloon and distal portion of the drug tube are outside the carrier tube, the distal portion of the drug tube takes on a relaxed radially expanded state.

2.-3. (canceled)

4. The drug eluting apparatus of claim 1, wherein the handle comprises an actuator, the actuator is configured to move the shaft relative to the carrier tube or to move the carrier tube relative to the shaft.

5. The drug eluting apparatus of claim 1, wherein the carrier tube is configured to be attached to a guiding catheter.

6. The drug eluting apparatus of claim 1, wherein, before the balloon is inflated, the distal portion of the drug tube is in a relaxed radially collapsed state, and when the balloon is inflated with the inflation fluid, the distal portion of the drug tube is expanded by the balloon into a deformed radially expanded state.

7. The drug eluting apparatus of claim 1, wherein, when the distal portion of the drug tube is in the relaxed radially expanded state, the distal portion of the drug tube extends in a helical shape.

8. The drug eluting apparatus of claim 6, wherein, when the distal portion of the drug tube is in the deformed radially expanded state, the distal portion of the drug tube extends in a helical shape.

9. The drug eluting apparatus of claim 1, wherein, when the distal portion of the drug tube is in the relaxed radially expanded state, the distal portion of the drug tube snakes circumferentially back and forth in a direction from a proximal end of the balloon to a distal end of the balloon.

10. The drug eluting apparatus of claim 6, wherein, when the distal portion of the drug tube is in the deformed radially expanded state, the distal portion of the drug tube snakes circumferentially back and forth in a direction from a proximal end of the balloon to a distal end of the balloon.

11. The drug eluting apparatus of claim 1, wherein, when the distal portion of the drug tube is in the relaxed radially expanded state, the distal portion of the drug tube comprises a plurality of branching drug tubes that are distributed over the exterior of the balloon.

12. The drug eluting apparatus of claim 6, wherein, when the distal portion of the drug tube is in the deformed radially expanded state, the distal portion of the drug tube comprises a plurality of branching drug tubes that are distributed over the exterior of the balloon.

13. The drug eluting apparatus of claim 11, wherein each of the branching drug tubes has an end portion that curls back on itself.

14. The drug eluting apparatus of claim 12, wherein each of the branching drug tubes has an end portion that curls back on itself.

15. The drug eluting apparatus of claim 1, wherein, when the distal portion of the drug tube is in the relaxed radially expanded state, the distal portion of the drug tube has a lattice shape.

16. The drug eluting apparatus of claim 6, wherein, when the distal portion of the drug tube is in the deformed radially expanded state, the distal portion of the drug tube has a lattice shape.

17. The drug eluting apparatus of claim 1, wherein the drug tube is made of nitinol or stainless steel.

18. The drug eluting apparatus of claim 1, wherein the balloon inflation lumen, the drug lumen, and the guidewire lumen are separate from each other.

19. The drug eluting apparatus of claim 1, wherein:
the drug lumen extends through the balloon inflation lumen; and
the guidewire lumen extends through the drug lumen.

20. The drug eluting apparatus of claim 1, wherein the shaft comprises an outer tube in which a shaft lumen is formed.

21. The drug eluting apparatus of claim 20, further comprising a balloon inflation tube in which the balloon inflation lumen is formed, the balloon inflation tube extending through the shaft lumen.

22. The drug eluting apparatus of claim 20, further comprising a guidewire tube in which the guidewire lumen is formed, the guidewire tube extending through the shaft lumen.

23. The drug eluting apparatus of claim 20, wherein a proximal portion of the drug tube extends through the shaft lumen, and the drug lumen is formed in the proximal portion of the drug tube.

24.-31. (canceled)

32. A method of delivering a drug into a vessel, the method comprising:
providing a drug eluting apparatus that comprises:
a handle comprising:
a balloon inflation port configured to receive an inflation fluid, and
a drug port configured to receive a drug;
a shaft connected to the handle, the shaft comprising:
a balloon inflation lumen configured to receive the inflation fluid from the balloon inflation port,
a drug lumen configured to receive the drug from the drug port, and
a guidewire lumen configured to receive a guidewire and allow the guidewire to extend out of a distal portion of the shaft;
a balloon attached to the distal portion of the shaft and configured to receive the inflation fluid from the balloon inflation lumen; and
a drug tube defining a distal portion of the drug lumen, the drug tube having a distal portion connected to an exterior of the balloon and comprising a plurality of holes configured to allow the drug to exit the drug tube;
inserting the distal portion of the shaft into a vessel;
inflating the balloon by providing the inflation fluid to the balloon via the balloon inflation lumen, which causes the distal portion of the drug tube to take a deformed radially expanded state; and
delivering the drug into the vessel via the drug lumen and the plurality of holes of the drug tube.

33. A method of delivering a drug into a vessel, the method comprising:
providing a drug eluting apparatus that comprises:
a handle comprising:
a balloon inflation port configured to receive an inflation fluid, and
a drug port configured to receive a drug;
a shaft connected to the handle, the shaft comprising:
a balloon inflation lumen configured to receive the inflation fluid from the balloon inflation port,
a drug lumen configured to receive the drug from the drug port, and
a guidewire lumen configured to receive a guidewire and allow the guidewire to extend out of a distal portion of the shaft;
a balloon attached to the distal portion of the shaft and configured to receive the inflation fluid from the balloon inflation lumen;
a carrier tube connected to the handle, the carrier tube being configured to allow the shaft to move therethrough, the carrier tube having a length that is greater than or equal to a length of the shaft; and
a drug tube defining a distal portion of the drug lumen, the drug tube having a distal portion connected to an exterior of the balloon and comprising a plurality of holes configured to allow the drug to exit the drug tube;
inserting the distal portion of the shaft into a vessel;
pushing the shaft or pulling the carrier tube so that the distal portion of the drug tube and the balloon are outside of the carrier tube, which causes the distal portion of the drug tube to take a relaxed radially expanded state;
inflating the balloon by providing the inflation fluid to the balloon via the balloon inflation tube; and
delivering the drug into the vessel via the drug tube and the plurality of holes of the drug tube.

34. The method of claim 33, the method further comprising:
before inserting the distal portion of the shaft into the vessel, inserting a guiding catheter into the vessel;
in the step of inserting the distal portion of the shaft into the vessel, inserting the distal portion of the shaft into the guiding catheter; and
in the step of pushing the shaft or pulling the carrier tube, pushing the shaft so that the distal portion of the drug tube and the balloon are outside of the guiding catheter.

35. A method of delivering a drug into a vessel, the method comprising:
providing a drug eluting apparatus that comprises:
a handle comprising:
a balloon inflation port configured to receive an inflation fluid, and
a drug port configured to receive a drug;
a shaft connected to the handle, the shaft comprising:
a balloon inflation lumen configured to receive the inflation fluid from the balloon inflation port,
a drug lumen configured to receive the drug from the drug port, and
a guidewire lumen configured to receive a guidewire and allow the guidewire to extend out of a distal portion of the shaft;
a balloon attached to the distal portion of the shaft and configured to receive the inflation fluid from the balloon inflation lumen;
a carrier tube connected to or connectable to the handle, the carrier tube configured to allow the shaft to move therethrough, the carrier tube configured to be attached to a guiding catheter; and
a drug tube defining a distal portion of the drug lumen, the drug tube having a distal portion connected to an exterior of the balloon and comprising a plurality of holes configured to allow the drug to exit the drug tube; wherein:
the carrier tube has a length that is approximately equal to an axial length of the distal portion of the drug tube;
inserting the distal portion of the shaft, the carrier tube, and the guiding catheter into a vessel;
pushing the shaft so that the distal portion of the drug tube and the balloon are outside of the carrier tube and the guiding catheter, which causes the distal portion of the drug tube to take a relaxed radially expanded state;
inflating the balloon by providing the inflation fluid to the balloon via the balloon inflation tube; and
delivering the drug into the vessel via the drug tube and the plurality of holes of the drug tube.

36.-38. (canceled)
